(19) United States
(12) Patent Application Publication
(10) Pub. No.: US 2026/0144239 A1
(43) Pub. Date: May 28, 2026

(54) Immune Cell-Engrafted Non-Human Animals and Non-Human Animal Models

(71) Applicant: LAPIX Therapeutics, Inc., Cambridge, MA (US)

(72) Inventors: Anas Fathallah, Wakefield, MA (US); Abdulraouf Ramadan, Malden, MA (US)

(73) Assignee: LAPIX Therapeutics, Inc., Cambridge, MA (US)

(21) Appl. No.: 19/122,068

(22) PCT Filed: Oct. 24, 2023
(86) PCT No.: PCT/US2023/077582

§ 371 Date: Apr. 17, 2025

Related U.S. Application Data
(60) Provisional application No. 63/419,115, filed on Oct. 25, 2022.
(60) Provisional application No. 63/386,007, filed on Dec. 5, 2022.

(51) Int. Cl. A01K 67/0271; A61K 49/00

(52) U.S. Cl. CPC A01K 67/0271; A61K 49/0008; A01K 2207/12; A01K 2207/15; A01K 2207/20; A01K 2227/105; A01K 2267/03

(57) ABSTRACT

The disclosure provides, in various embodiments, non-human animals that comprise a human immune cell engraftment and/or functional human immune system. The disclosure also provides, in various embodiments, methods of generating said non-human animals. The disclosure also provides, in various embodiments, methods of determining immunogenicity of antigens or an immunogenic fragment thereof or antigenic therapies and/or identifying agents that modulate immune response.

### CROSS-REFERENCE TO RELATED APPLICATIONS

This application claims the benefit of priority of U.S. Provisional Patent Application No. 63/419,115, filed Oct. 25, 2022, and U.S. Provisional Patent Application No. 63/386,007, filed Dec. 5, 2022, each of which is incorporated by reference herein in its entirety.

### BACKGROUND

The use of various therapeutics in clinical settings has been complicated by unwanted immunogenic responses, including production of antidrug antibodies (ADAs) in response to protein-based therapeutics and gene therapy. Immunogenicity not only reduces utility and efficacy of a therapy, but also can be life-threatening. There is currently no preclinical model that can predict the human immune response to therapy, such as monoclonal antibodies, gene therapy, protein replacement therapy, etc.

Accordingly, there is a need for methods and animal models for predicting clinical human immune responses.

### SUMMARY

Provided herein in some aspects are non-human animals comprising a functional human immune system, as well as methods of making and using the same, e.g., for determining the immunogenicity of an agent, such as a vaccine, enzyme replacement therapy, or gene therapy, and/or identifying or assessing an effect of an agent, such as an immune modulator drug, that modulates immune response.

In one aspect, a non-human animal disclosed herein:
a) lacks endogenous mature T cells; and
b) comprises an engrafted population of human mononuclear cells, human polymorphonuclear leukocytes, or both, wherein the engraftment lacks cancer cells (e.g., human cancer cells).

In one aspect, a non-human animal disclosed herein:
a) lacks endogenous mature T cells; and
b) comprises a functional human immune system generated from an engraftment of a population of human mononuclear cells, human polymorphonuclear leukocytes, or both, wherein the engraftment lacks cancer cells (e.g., human cancer cells).

In another aspect, a method disclosed herein comprises engrafting a population of human mononuclear cells, human polymorphonuclear leukocytes, or both into a non-human animal lacking endogenous mature T cells, wherein the engraftment lacks human cells (e.g., human cancer cells).

In another aspect, a method disclosed herein comprises engrafting a population of human mononuclear cells, human polymorphonuclear leukocytes, or both into a non-human animal lacking endogenous mature T cells, wherein the engraftment lacks cancer cells (e.g., human cancer cells), and wherein the engraftment produces a functional human immune system in the non-human animal.

In another aspect, a method disclosed herein comprises:
for n individual non-human animals lacking endogenous mature T cells, engrafting a population of human mononuclear cells, human polymorphonuclear leukocytes, or both from a n^{th} human into a n^{th} non-human animal, wherein n is an integer ≥2,
thereby generating a non-human animal model comprising two or more individual non-human animals,
wherein the engraftments lack cancer cells (e.g., human cancer cells) and produce a functional human immune system in the individual non-human animals.

In another aspect, a method disclosed herein comprises:
administering an antigen or an immunogenic fragment thereof, or an antigenic therapy to any one or more non-human animals described herein or one or more non-human animals of a non-human animal model disclosed herein; and
detecting an immune response to the antigen or the immunogenic fragment thereof, or the antigenic therapy in the one or more non-human animals,
thereby determining immunogenicity of the antigen or the immunogenic fragment thereof, or the antigenic therapy.

In another aspect, a method disclosed herein comprises:
administering an antigen or an immunogenic fragment thereof, or an antigenic therapy to any one or more non-human animals described herein or one or more non-human animals of a non-human animal model disclosed herein;
administering an agent suspected to modulate immune response of the one or more non-human animals to the antigen or the immunogenic fragment thereof, or the antigenic therapy; and
detecting an immune response to the antigen or the immunogenic fragment thereof, or the antigenic therapy in the one or more non-human animals,
thereby identifying an agent for modulating immune response.

The non-human animals described herein, in some embodiments, have a human donor's B cells, T cells, macrophages, monocytes, and/or NK cells, meaning that the non-human animal produces human antibodies. In some embodiments, antibodies produced by the animal are only human antibodies. With a large enough donor pool, the model can predict the clinical immunogenicity rate of biologics. Multiple non-human animals engrafted with cells from the same donor (e.g., a healthy human donor) can be used to test the effect of products and product-related attributes (e.g., formulation, aggregates, route of administration, etc.) on immunogenicity.

Unlike humanized or genetically engineered mice, the non-human animals described herein can develop a donor-derived human antibody response, and two non-human animals each comprising engraftments from two different donors may have different responses. In this way, the non-human animals described herein can recapitulate a human immune response (such as a response specific to a particular human donor).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 illustrates the workflow of Example 1, where a NRGS mouse was conditioned on Day −1, engrafted with T-cell depleted peripheral blood mononuclear cells (PBMCs) from a human on Day 0, and engrafted with CD4⁺ T cells from the human on Day 28. For some applications (e.g., oncology, viral infection, vaccine, etc.), the mouse is further engrafted with CD8⁺ T cells about 14 days after engraftment with CD4⁺ T cells (e.g., on Day 42) (not shown).

FIG. 2A is representative flow cytometry plots, and shows co-expression of both mouse and human CD45 but only human CD3 in the Day 28 engrafted mice from Example 1.

FIG. 2B is representative flow cytometry plots, and shows that in engrafted mice prepared in accordance with Example 1, mouse CD45⁺ cells were CD4⁻ and CD19⁻, and human CD45⁺ cells comprise CD4⁺ cells and CD19⁺ cells.

FIG. 3A is a box plot, and shows the percentages of human CD45⁺ cells in mice engrafted with cells from 10 healthy human donors (human donor (HD)15-21 and HD23-25) in accordance with Example 1.

FIG. 3B is a box plot, and shows the percentage of human CD45⁺/CD4⁺ T cells in mice engrafted with cells from 10 healthy human donors (human donor (HD)15-21 and HD23-25) in accordance with Example 1.

FIG. 3C is a box plot, and shows the percentages of human CD45⁺/CD19⁺ B cells in mice engrafted with cells from 10 healthy human donors (human donor (HD)15-21 and HD23-25) in accordance with Example 1.

FIG. 4A is a representative flow cytometry plot, and shows the percentages of mouse and human hematopoietic (CD45⁺) cells on Day 28 in one of four mice engrafted with cells from a human donor in accordance with Example 1. Day 28 refers to 28 days after the day preconditioned mice received T-cell depleted PBMCs.

FIG. 4B is a representative flow cytometry plot, and shows the percentages of mouse and human hematopoietic (CD45⁺) cells on Day 183 in one of four mice engrafted with cells from a human donor in accordance with Example 1. Day 183 refers to 183 days after the day preconditioned mice received T-cell depleted PBMCs.

FIG. 4C is a bar graph, and shows the percentages of human hematopoietic (CD45⁺) cells on Days 28 and 183 in mice engrafted with cells from two human donors in accordance with Example 1.

FIG. 5A is a representative flow cytometry plot, and shows the percentages of mouse and human CD4⁺ cells (T cells) on Day 28 in one of four mice engrafted with cells from two human donors in accordance with Example 1.

FIG. 5B is a representative flow cytometry plot, and shows the percentages of mouse and human CD4⁺ cells (T cells) on Day 183 in one of four mice engrafted with cells from two human donors in accordance with Example 1.

FIG. 5C is a bar graph, and shows the percentages of human CD45⁺/CD4⁺ cells on Days 28 and 183 in mice engrafted with cells from two human donors in accordance with Example 1.

FIG. 6A is a representative flow cytometry plot, and shows the percentages of mouse and human CD19⁺ cells (B cells) on Day 28 in one of four mice engrafted with cells from two human donors in accordance with Example 1.

FIG. 6B is a representative flow cytometry plot, and shows the percentages of mouse and human CD19⁺ cells (B cells) on Day 183 in one of four mice engrafted with cells from two human donors in accordance with Example 1.

FIG. 6C is a bar graph, and shows the percentages of human CD45⁺/CD19⁺ cells on Days 28 and 183 in mice engrafted with cells from two human donors in accordance with Example 1.

FIG. 7A is flow cytometry plots, and shows the CD8⁺/CD4⁺ ratio in engrafted mice prepared in accordance with Example 1.

FIG. 7B is a box plot, and shows percentages of hCD3⁺/hCD8⁺ cells in engrafted mice prepared in accordance with Example 1.

FIG. 7C is a bar graph, and summarizes the percentage CD45⁺/CD3⁺/CD8⁺ and CD45⁺/CD3⁺/CD4⁺ data from FIG. 7A.

FIG. 7D is flow cytometry plots, and shows the percentage of human myeloid cells on day 56 post-engraftment in mice engrafted with cells from two human donors in accordance with Example 1.

FIG. 7E is flow cytometry plots, and shows the percentage of human myeloid cells on day 185 post-engraftment in mice engrafted with cells from two human donors in accordance with Example 1.

FIG. 7F is flow cytometry plots, and shows the percentage of human NK cells seven months after protocol initiation in mice engrafted with cells from two human donors in accordance with Example 1.

FIG. 8A shows immunogenicity rate against human FVIII, based on titers of human anti-FVIII antibodies in engrafted mice and C57B6 (control) mice administered two doses of FVIII or GAA (as a control).

FIG. 8B shows immunogenicity rate against mouse FVIII, based on titers of mouse anti-FVIII antibodies in engrafted mice and C57B6 (control) mice administered two doses of FVIII or GAA (as a control).

FIG. 8C shows immunogenicity rate against human GAA, based on titers of human anti-GAA antibodies in engrafted mice and C57B6 (control) mice administered two doses of GAA or FVIII (as a control).

FIG. 8D shows immunogenicity rate against mouse GAA, based on titers of mouse anti-GAA antibodies in engrafted mice and C57B6 (control) mice administered two doses of GAA or FVIII (as a control).

FIG. 9A is a flow cytometry plot, and shows the percentage of CD20⁺/CD19⁺ cells in a representative engrafted mouse that developed antibodies to FVIII in accordance with Example 3A.

FIG. 9B is a flow cytometry plot, and shows the percentage of CD20⁺/CD19⁺ cells in a representative engrafted mouse that developed antibodies to AAV9 in accordance with Example 3D.

FIG. 9C is a flow cytometry plot, and shows the percentage of CD20⁺/CD19⁺ cells in a representative engrafted mouse that developed antibodies to GAA in accordance with Example 3B.

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

When introducing elements disclosed herein, the articles “a,” “an,” “the,” and “said” are intended to mean that there are one or more of the elements. Further, the one or more elements may be the same or different. Thus, for example, unless the context clearly indicates otherwise, “an engraftment” includes a single engraftment, and two or more engraftments. Further the two or more engraftments can be the same or different as, for example, in embodiments wherein a first engraftment comprises a population of T cell-depleted PBMCs and a second engraftment comprises a population of T cells).

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification. When “about” precedes a range, as in “about 24-96 hours,” the term “about” should be read as applying to both of the given values of the range, such that “about 24-96 hours” means about 24 hours to about 96 hours.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Antigen,” as used herein, refers to any substance that can be recognized by the immune system. “Antigen” broadly encompasses proteins, such as enzymes, peptides, such as polypeptides, carbohydrates, such as polysaccharides, haptens, nucleic acids such as polynucleotides, and grafts. An antigen can be a self-antigen, an antigen produced, under normal conditions or as part of a disorder, by the body, or a foreign antigen, a non-self-antigen. Examples of self-antigens include self-antigens associated with autoimmune disorders. Examples of foreign antigens include antigenic therapies (e.g., therapeutic proteins, gene therapies, cellular therapies), allergens and alloantigens.

### Methods of Generating Non-Human Animals and Non-Human Animal Models

Provided herein, among other things, are methods of generating non-human animals and non-human animal models comprising a functional human immune system.

In one aspect, a method disclosed herein comprises engrafting a population of human mononuclear cells, human polymorphonuclear leukocytes, or both into a non-human animal lacking endogenous mature T cells (e.g., lacking endogenous CD8+ “killer” (cytotoxic) T cells and/or endogenous CD4+ “helper” T cells). In some embodiments, the engraftment lacks cancer cells (e.g., human cancer cells). In some embodiments, the engraftment produces a functional human immune system in the non-human animal. In some embodiments, the population of human mononuclear cells, human polymorphonuclear leukocytes, or both, is from at least one human (e.g., at least one healthy human). In some embodiments, the population of human mononuclear cells, human polymorphonuclear leukocytes, or both, is from two or more humans (e.g., two or more healthy humans).

In one aspect, a method disclosed herein comprises: for n individual non-human animals lacking endogenous mature T cells (e.g., lacking endogenous CD8+ “killer” (cytotoxic) T cells and/or endogenous CD4+ “helper” T cells), engrafting a population of human mononuclear cells, human polymorphonuclear leukocytes, or both from a n^{th} human into a n^{th} non-human animal, wherein n is an integer ≥2, thereby generating a non-human animal model comprising two or more individuals. In some embodiments, the engraftments lack cancer cells (e.g., human cancer cells). In some embodiments, the engraftments produce a functional human immune system(s) in the individual non-human animals.

In another aspect, a method disclosed herein comprises: for n individual non-human animals lacking endogenous mature T cells (e.g., lacking endogenous CD8+ “killer” (cytotoxic) T cells and/or endogenous CD4+ “helper” T cells), engrafting a population of human mononuclear cells, human polymorphonuclear leukocytes, or both from at least one human into the n individual non-human animals, wherein n is an integer ≥2, thereby generating a non-human animal model comprising two or more individual non-human animals. In some embodiments, the engraftments lack cancer cells (e.g., human cancer cells). In some embodiments, the engraftments produce a functional human immune system(s) in the individual non-human animals. In some embodiments, the at least one human is one human. In some embodiments, the at least one human is two or more humans.

### Non-Human Animals

In some embodiments, a non-human animal disclosed herein is a mammal, such as a primate, a pig, a cow, a bull, a horse, a sheep, a goat, a rabbit, a dog, a cat, a rat or a mouse. In some embodiments, a non-human animal is a laboratory animal. In some embodiments, a laboratory animal is a rodent. In some embodiments, a rodent is a rat. In some embodiments, a rat is a transgenic rat.

In some embodiments, a rodent is a mouse. In some embodiments, a mouse is a mutant, transgenic, knockdown, and/or knockout mouse. In some embodiments, a mouse is female. In some embodiments, a mouse is male. In some embodiments, a mouse (e.g., a mutant, transgenic, knockdown, and/or knockout mouse) is about 2-12 months old, e.g., about 2 months old, about 3 months old, about 4 months old, about 5 months old, about 6 months old, about 7 months old, about 8 months old, about 9 months old, about 10 months old, about 11 months old, about 12 months old, about 2-11 months old, about 2-10 months old, about 2-9 months old, about 2-8 months old, about 2-7 months old, about 2-6 months old, about 2-5 months old, about 2-4 months old, about 2-3 months old, about 3-12 months old, about 3-11 months old, about 3-10 months old, about 3-9 months old, about 3-8 months old, about 3-7 months old, about 3-6 months old, about 3-5 months old, about 3-4 months old, about 4-12 months old, about 4-11 months old, about 4-10 months old, about 4-9 months old, about 4-8 months old, about 4-7 months old, about 4-6 months old, about 4-5 months old, about 5-12 months old, about 5-11 months old, about 5-10 months old, about 5-9 months old, about 5-8 months old, about 5-7 months old, about 5-6 months old, about 6-12 months old, about 6-11 months old, about 6-10 months old, about 6-9 months old, about 6-8 months old, about 6-7 months old, about 7-12 months old, about 7-11 months old, about 7-10 months old, about 7-9 months old, about 7-8 months old, about 8-12 months old, about 8-11 months old, about 8-10 months old, about 8-9 months old, about 9-12 months old, about 9-11 months old, about 9-10 months old, about 10-12 months old, about 10-11 months old, or about 11-12 months old. In some embodiments, a mouse (e.g., a mutant, transgenic, knockdown, and/or knockout mouse) is about 2-6 months old.

The non-human animals described herein lack endogenous mature T cells. In some embodiments, a non-human animal lacks endogenous mature T cells and comprises an engrafted population of human mononuclear cells, human polymorphonuclear leukocytes, or both. In some embodiments, the human mononuclear cells, human polymorphonuclear leukocytes, or both are from at least one human (e.g., one human; two or more humans, such as a population of humans). In another embodiment, a non-human animal lacks endogenous mature T cells, and comprises a functional human immune system generated from an engraftment of a population of human mononuclear cells, human polymorphonuclear leukocytes, or both. In particular embodiments, the engraftment lacks cancer cells (e.g., human cancer cells).

Thus, in some embodiments, the non-human animal comprises one or more human lymphocyte cell types, one or more human myeloid cell types, or any combination thereof. In some embodiments, the one or more human lymphocyte cell types comprise human T cells, human B cells, human NK cells, or any combination thereof. In some embodiments, the one or more human myeloid cell types comprise human monocytes, human macrophages, or both. In some particular embodiments, the non-human animal comprises (e.g., is engrafted with) human T cells, human B cells, human NK cells, human monocytes, and human macrophages, such as from one or more healthy human donors.

In some embodiments, the non-human animal comprises both endogenous CD45+ cells and human CD45+ cells. In some embodiments, the human T cells comprise human CD4+ T cells, human CD8+ T cells, or both. In some embodiments, the human B cells comprise human CD19+ B cells. In some embodiments, the human NK cells comprise human CD56+ NK cells. In some embodiments, the one or more human myeloid cell types comprises CD14+/CD11b+ cells.

In particular embodiments:
a) less than 15%, less than 10%, less than 5%, or less than 1% of CD3 cells in the non-human animal are endogenous CD3 cells;
b) the non-human animal expresses human IL-3, human GM-CSF, and human SF;
c) the non-human animal lacks Rag1 expression, function, or both; and/or
d) the non-human animal lacks IL2rγ expression, function, or both.

In a particular embodiment, the non-human animal lacks endogenous mature T cells, endogenous mature B cells, and endogenous mature NK cells; comprises an engrafted population of human mononuclear cells, human polymorphonuclear leukocytes, or both, wherein the engraftment lacks human cancer cells, and wherein the population of human mononuclear cells comprises human T cells, human B cells, human NK cells, human monocytes, and human macrophages; expresses human IL-3, human GM-CSF, and human SF; lacks Rag1 expression, function, or both; and lacks IL2rγ expression, function, or both.

In another particular embodiment, the non-human animal lacks endogenous mature T cells, endogenous mature B cells, and endogenous mature NK cells; comprises an engrafted population of human mononuclear cells, wherein the engraftment lacks human cancer cells, and wherein the population of human mononuclear cells comprises human T cells, human B cells, human NK cells, human monocytes, and human macrophages; expresses human IL-3, human GM-CSF, and human SF; lacks Rag1 expression, function, or both; and lacks IL2rγ expression, function, or both.

In another particular embodiment, the non-human animal lacks endogenous mature T cells, endogenous mature B cells, and endogenous mature NK cells; comprises an engrafted population of human polymorphonuclear leukocytes, wherein the engraftment lacks human cancer cells; expresses human IL-3, human GM-CSF, and human SF; lacks Rag1 expression, function, or both; and lacks IL2rγ expression, function, or both.

In some embodiments, less than 15% of CD3 cells in a non-human animal comprising a functional human immune system are endogenous CD3 cells, for example, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2% or less than 0.1%, or 0% of CD3 cells in the non-human animal comprising a functional human immune system are endogenous CD3 cells.

In some embodiments, a non-human animal disclosed herein has severe impairments in endogenous T-, B- and/or natural killer (NK)-cell development. In some embodiments, a non-human animal lacks endogenous T-, endogenous B-, and endogenous NK-cell development (e.g., via gene knockdown and/or knockout). In some embodiments, a non-human animal disclosed herein lacks endogenous mature B cells, endogenous mature NK cells, or both. In some embodiments, a non-human animal disclosed herein lacks endogenous mature B cells. In some embodiments, a non-human animal disclosed herein lacks endogenous mature NK cells. In some embodiments, a non-human animal disclosed herein lacks endogenous mature B cells and endogenous mature NK cells.

In some embodiments, a non-human animal described herein lacks endogenous T cells, endogenous B cells, endogenous NK cells, or a combination thereof. In some embodiments, a non-human animal described herein lacks endogenous T cells (e.g., endogenous CD4+ T cells and/or endogenous CD8+ T cells). In some embodiments, a non-human animal described herein lacks endogenous B cells (such as endogenous CD19+ B cells). In some embodiments, a non-human animal described herein lacks endogenous T cells and endogenous B cells. In some embodiments, a non-human animal described herein lacks endogenous NK cells (such as endogenous NK1.1 cells (e.g., in a mouse, such as a C57 BL/6 mouse)). In some embodiments, a non-human animal described herein lacks endogenous T cells and endogenous NK cells. In some embodiments, a non-human animal described herein lacks endogenous T cells, endogenous B cells, and endogenous NK cells.

In some embodiments, a non-human animal described herein comprises antigen presenting cells that lack major histocompatibility complex (MHC) class I expression, function, or both, and/or that lack MHC class II expression, function, or both.

In some embodiments, a non-human animal disclosed herein:
expresses human interleukin-3 (IL-3) (e.g., comprises cells that express human IL-3);
expresses human granulocyte/macrophage-stimulating factor (GM-CSF) (e.g., comprises cells that express human GM-CSF);
expresses human steel factor (SF) (e.g., comprises cells that express human SF);
lacks recombination activating 1 (Rag1) expression, function, or both;
lacks IL2 receptor common gamma chain (IL2rγ) expression, function, or both;
lacks recombination activating 2 (Rag2) expression, function, or both;
lacks DNA-dependent protein kinase (DNA-PK) expression, function, or both;
lacks β2 microglobulin (B2m) expression, function, or both;
lacks perforin (Prf1) expression, function, or both;
lacks T-cell receptor (TCR) α chain expression, function, or both; or
lacks major histocompatibility complex (MHC) class II expression, function, or both, or
any combination of the foregoing.

In some embodiments, a non-human animal disclosed herein:
expresses human IL-3 (e.g., comprises cells that express IL-3);
expresses human GM-CSF (e.g., comprises cells that express GM-CSF);
expresses human SF (e.g., comprises cells that express SF);
lacks Rag1 expression, function, or both; and
lacks IL2rγ expression, function, or both.

In some embodiments, a non-human animal disclosed herein is a NRGS mouse (e.g., commercially available from The Jackson Laboratory, Bar Harbor, ME), a NSGS mouse (e.g., commercially available from The Jackson Laboratory, Bar Harbor, ME), a huNOG mouse e.g., commercially available from Taconic Biosciences, Albany, NY), or a scid mouse (e.g., commercially available from The Jackson Laboratory, Bar Harbor, ME). In some embodiments, a non-human animal disclosed herein is a NRGS mouse. In some embodiments, a non-human animal disclosed herein is a NSGS mouse.

In some embodiments, a non-human animal disclosed herein (e.g., a NRGS mouse or a NSGS mouse) expresses a cytokine that promotes the growth and/or development of polynuclear cells and/or lymphocytes. In some embodiments, a non-human animal disclosed herein (e.g., a NRGS mouse or a NSGS mouse) expresses human interleukin-5 (IL-5), human interleukin-7 (IL-7), or both.

### Conditioning

Conditioning can be used to prepare a non-human animal for engraftment. Without being bound by theory, conditioning can be used, for example, to eliminate endogenous hemopoietic cells in a non-human animal and/or to increase proliferation and survival of mononuclear cells and polymorphonuclear leukocytes.

Thus, in some embodiments, a method disclosed herein (e.g., of generating a non-human animal or non-human animal model) further comprises conditioning a non-human animal. It will be appreciated that conditioning typically occurs prior to the engrafting.

In some embodiments, a non-human animal disclosed herein (e.g., a NRGS mouse or a NSGS mouse) is conditioned with radiation, chemotherapy, or a combination thereof.

In some embodiments, a non-human animal disclosed herein (e.g., a mouse, such as a NRGS mouse or a NSGS mouse) is conditioned with radiation. In some embodiments, a non-human animal is sublethally irradiated. In some embodiments, a non-human animal (e.g., a mouse, such as a NRGS mouse or a NSGS mouse) is conditioned with radiation at a dose of about 140 cGy to about 310 cGy, for example, at a dose of about 140 cGy, about 150 cGy, about 160 cGy, about 170 cGy, about 180 cGy, about 190 cGy, about 200 cGy, about 210 cGy, about 220 cGy, about 230 cGy, about 240 cGy, about 250 cGy, about 260 cGy, about 270 cGy, about 280 cGy, about 290 cGy, about 300 cGy, about 310 cGy, about 150-310 cGy, about 150-300 cGy, about 160-300 cGy, about 160-290 cGy, about 170-290 cGy, about 170-280 cGy, about 180-280 cGy, about 180-270 cGy, about 190-270 cGy, about 190-260 cGy, about 200-260 cGy, about 200-250 cGy, about 210-250 cGy, about 210-240 cGy, about 220-240 cGy, or about 220-230 cGy.

In some embodiments, a non-human animal (e.g., a NRGS mouse) is conditioned with radiation at a dose of about 250-350 cGy, for example, about 250 cGy, about 260 cGy, about 270 cGy, about 280 cGy, about 290 cGy, about 300 cGy, about 310 cGy, about 320 cGy, about 330 cGy, about 340 cGy, about 350 cGy, about 250-340 cGy, about 260-340 cGy, about 260-330 cGy, about 270-330 cGy, about 270-320 cGy, about 280-320 cGy, about 280-310 cGy, about 290-310 cGy, or about 290-300 cGy. In some embodiments, a non-human animal (e.g., a NRGS mouse) is conditioned with radiation at a dose of about 300 cGy.

In some embodiments, a non-human animal (e.g., a NSGS mouse) is conditioned with radiation at a dose of about 100-200 cGy, for example, about 100 cGy, about 110 cGy, about 120 cGy, about 130 cGy, about 140 cGy, about 150 cGy, about 160 cGy, about 170 cGy, about 180 cGy, about 190 cGy, about 200 cGy, about 100-190 cGy, about 110-190 cGy, about 110-180 cGy, about 120-180 cGy, about 120-170 cGy, about 130-170 cGy, about 130-160 cGy, about 140-160 cGy, or about 140-150 cGy. In some embodiments, a non-human animal (e.g., a NSGS mouse) is conditioned with radiation at a dose of about 150 cGy.

In some embodiments, a non-human animal (e.g., a NRGS or NSGS mouse) is conditioned with a single dose of radiation. In some embodiments, a non-human animal (e.g., a NRGS or NSGS mouse) is conditioned with a single dose of radiation about 24 to about 96 hours before engrafting of a population of human mononuclear cells, human polymorphonuclear leukocytes, or both, into the non-human animal, for example, about 24 hours, about 48 hours, about 72 hours, about 96 hours, about 24 to about 72 hours, about 24 to about 48 hours, about 48 to about 72 hours, about 48 to about 96 hours or about 72 to about 96 hours before engrafting a population of human mononuclear cells, human polymorphonuclear leukocytes, or both. In some embodiments, a non-human animal (e.g., a NRGS or NSGS mouse) is conditioned with a single dose about 24 hours before engrafting of a population of human mononuclear cells, human polymorphonuclear leukocytes, or both, into the non-human animal.

In some embodiments, a non-human animal (e.g., a mouse, such as a NRGS or NSGS mouse) is conditioned with two doses of radiation (e.g., two equal doses, within 6 hours of one another). In some embodiments, a non-human animal (e.g., a mouse, such as a NRGS or NSGS mouse) is conditioned with two doses about 24 to about 96 hours before engrafting of a population of human mononuclear cells, human polymorphonuclear leukocytes, or both, into the non-human animal, for example, about 24 hours, about 48 hours, about 72 hours, about 96 hours, about 24 to about 72 hours, about 24 to about 48 hours, about 48 to about 72 hours, about 48 to about 96 hours or about 72 to about 96 hours before engrafting a population of human mononuclear cells, human polymorphonuclear leukocytes, or both. In some embodiments, a non-human animal (e.g., a mouse, such as a NRGS or NSGS mouse) is conditioned with two doses of radiation (e.g., two equal doses, within 6 hours of one another). In some embodiments, a non-human animal (e.g., a mouse, such as a NRGS or NSGS mouse) is conditioned with two doses about 24 hours before engrafting of a population of human mononuclear cells, human polymorphonuclear leukocytes, or both, into the non-human animal. In some embodiments, a non-human animal (e.g., a mouse, such as a NRGS or NSGS mouse) is conditioned with two doses (e.g., two equal doses) of radiation about 48 hours and about 24 hours before engrafting of a population of human mononuclear cells, human polymorphonuclear leukocytes, or both, into the non-human animal.

In some embodiments, a non-human animal (e.g., a mouse, such as a NRGS or NSGS mouse) is conditioned with a chemotherapy. In some embodiments, the chemotherapy comprises an alkylating agent (e.g., busulfan and/or cyclophosphamide), an immunosuppressant (e.g., cyclophosphamide and/or cyclosporine), or a combination thereof. In some embodiments, chemotherapy comprises busulfan, cyclophosphamide, or cyclosporine, or any combination of the foregoing. In some embodiments, the chemotherapy comprises busulfan (e.g., about 25 mg/kg busulfan).

In some embodiments, a non-human animal (e.g., a mouse, such as a NRGS or NSGS mouse) is conditioned with a chemotherapy about 24 to about 96 hours before engrafting of a population of human mononuclear cells, human polymorphonuclear leukocytes, or both, into the non-human animal, for example, about 24 hours, about 48 hours, about 72 hours, about 96 hours, about 24 to about 72 hours, about 24 to about 48 hours, about 48 to about 72 hours, about 48 to about 96 hours or about 72 to about 96 hours before engrafting a population of human mononuclear cells, human polymorphonuclear leukocytes, or both. In some embodiments, a non-human animal (e.g., a mouse, such as a NRGS or NSGS mouse) is conditioned with a chemotherapy about 24 hours before engrafting a population of human mononuclear cells, human polymorphonuclear leukocytes, or both. In some embodiments, a non-human animal (e.g., a mouse, such as a NRGS or NSGS mouse) is conditioned with a chemotherapy about 48 hours before engrafting of a population of human mononuclear cells, human polymorphonuclear leukocytes, or both, into the non-human animal. In some embodiments, a non-human animal (e.g., a mouse, such as a NRGS or NSGS mouse) is conditioned with a chemotherapy about 24 hours and about 48 hours before engrafting of a population of human mononuclear cells, human polymorphonuclear leukocytes, or both, into the non-human animal.

Other conditioning therapies useful in the animals and methods disclosed herein, e.g., to reduce relapse and/or rejection of an engraftment are known to those skilled in the art.

In some embodiments, a non-human animal (e.g., a mouse, such as a NRGS or NSGS mouse) is conditioned with radiation and chemotherapy, for example, according to any of the embodiments for conditioning described herein. In some embodiments, a non-human animal (e.g., a mouse, such as a NRGS or NSGS mouse) is conditioned with radiation, for example, according to any of the embodiments for conditioning described herein. In some embodiments, a non-human animal (e.g., a mouse, such as a NRGS or NSGS mouse) is conditioned with a chemotherapy, for example, according to any of the embodiments for conditioning described herein. In some embodiments, a non-human animal (e.g., a mouse, such as a NRGS or NSGS mouse) is conditioned with radiation and a chemotherapy, for example, according to any of the embodiments for conditioning described herein.

In some embodiments, a method disclosed herein comprises conditioning a non-human animal, e.g., with radiation, a chemotherapy, or a combination thereof, for example, according to any of the embodiments for conditioning described herein.

### Humans

In some embodiments, a human disclosed herein (e.g., a human donor) is a healthy human (e.g., does not have any significant known health problem; does not suffer from a disease, disorder or condition of the immune system; and/or does not suffer from cancer). In some embodiments, a human is not diagnosed with or suspected of having a disease or condition, non-limiting examples of which include a cancer, an infection (e.g., a viral infection of human immunodeficiency virus (HIV), hepatitis C virus (HCV), or hepatitis B virus (HBV)), hypertension, allergy, an autoimmune disorder, diabetes mellitus, a pulmonary disease, asthma, or any combination of the foregoing. In some embodiments, a human disclosed herein is not taking any medication.

In some embodiments, a human (e.g., a healthy human donor) is a female. In some embodiments, a human (e.g., a healthy human donor) is a male.

In some embodiments, a human (e.g., a healthy human donor) is between the ages of 18-85 years, for example, 18-75 years, 18-65 years, 18-55 years, 55-85 years, 55-75 years, 55-65 years, 65-85 years, 65-75 years, or 75-85 years. In some embodiments, a human (e.g., a healthy human donor) is between the ages of 2-12 years, for example, 2-10 years, 2-8 years, 2-6 years, 2-5 years, 2-4 years, 4-12 years, 4-10 years, 4-8 years, 4-6 years, 4-5 years, 5-12 years, 5-10 years, 5-8 years, 5-6 years, 6-12 years, 6-10 years, 6-8 years, 8-12 years, or 8-10 years. In some embodiments, a human (e.g., a healthy human donor) is between the ages of 6 months to 17 years, for example, 6 months to 16 years, 6 months to 14 years, 6 months to 12 years, 6 months to 10 years, 6 months to 8 years, 6 months to 6 years, 6 months to 4 years, 6 months to 2 years, 1-17 years, 1-16 years, 1-14 years, 1-12 years, 1-10 years, 1-8 years, 1-6 years, 1-4 years, 1-2 years, 2-17 years, 2-16 years, 2-14 years, 2-12 years, 2-10 years, 2-8 years, 2-6 years, 2-4 years, 4-17 years, 4-16 years, 4-14 years, 4-12 years, 4-10 years, 4-8 years, 4-6 years, 6-17 years, 6-16 years, 6-14 years, 6-12 years, 6-10 years, 6-8 years, 8-17 years, 8-16 years, 8-14 years, 8-12 years, 8-10 years, 10-17 years, 10-16 years, 10-14 years, 10-12 years, 12-17 years, 12-16 years, 12-14 years, 14-17 years, or 14-16 years.

In some embodiments, a human (e.g., a healthy human donor) is ≥6 months, for example, ≥7 months, ≥8 months, ≥9 months, ≥10 months, ≥11 months, ≥1 year, ≥2 years, ≥3 years, ≥4 years, ≥5 years, ≥6 years, ≥7 years, ≥8 years, ≥9 years, ≥10 years, ≥11 years, ≥12 years, ≥13 years, ≥14 years, ≥15 years, ≥16 years, ≥17 years, or ≥18 years. In some embodiments, a human (e.g., a healthy human donor) is ≥2 years. In some embodiments, a human (e.g., a healthy human donor) is ≥12 years. In some embodiments, a human (e.g., a healthy human donor) is ≥18 years.

In some embodiments, a human (e.g., a healthy human donor) is about 6 months to about 17 years of age or is about 18 years or older.

In some embodiments, a human (e.g., a healthy human donor) is alive. In some embodiments, a human (e.g., a healthy human donor) is deceased.

In some embodiments, a human (e.g., a healthy human donor) received a treatment to mobilize stem cells. For example, certain treatments (such as a colony-stimulating factor or a chemotherapy) can increase the number of stem cells in the peripheral blood (a process known as “mobilization”), e.g., prior to collection of cells from a human for engraftment into a non-human animal as disclosed herein. In some embodiments, stem cell mobilization treatment comprises administration of a granulocyte colony-stimulating factor (G-CSF).

### Human Mononuclear Cells & Polymorphonuclear Leukocytes

In some embodiments, a population of human mononuclear cells, human polymorphonuclear leukocytes, or both is a population of human mononuclear cells. Mononuclear cells comprise blood cells that have a single, round nucleus, such as lymphocytes (such as T cells, B cells, and NK cells) and myeloid cells (such as monocytes and macrophages). Mononuclear cells may be isolated from circulating blood (peripheral blood mononuclear cells (PBMCs)), the umbilical cord, spleen, and/or bone marrow, such as by any suitable means known in the art. Accordingly, in some embodiments, a population of human mononuclear cells comprises peripheral blood mononuclear cells (PBMCs, e.g., G-CSF mobilized PBMCs), umbilical cord blood mononuclear cells (CB-MNCs, e.g., G-CSF mobilized CB-MNCs), bone marrow mononuclear cells (BMNCs, e.g., G-CSF mobilized BMNCs), or a combination thereof. In some embodiments, a population of human mononuclear cells comprises peripheral blood mononuclear cells (PBMCs, e.g., G-CSF mobilized PBMCs). In particular embodiments, the population of human mononuclear cells comprises T cells, B cells, monocytes, macrophages, and NK cells.

In some embodiments, a population of human mononuclear cells comprises about 5×10² to about 1×10⁹ mononuclear cells, for example, about 5×10²-3×10⁸, 5×10²-1×10⁸, about 5×10²-3×10⁷, about 5×10²-1×10⁷, about 5×10³-1×10⁹, about 5×10³-3×10⁸, about 5×10³-1×10⁸, about 5×10³-3×10⁷, about 5×10³-1×10⁷, about 5×10⁴-1×10⁹, about 5×10⁴-3×10⁸, about 5×10⁴-1×10⁸, about 5×10⁴-3×10⁷, about 5×10⁴-1×10⁷, about 5×10⁵-1×10⁹, about 5×10⁵-3×10⁸, about 5×10⁵-1×10⁸, about 5×10⁵-3×10⁷, about 5×10⁵-1×10⁷, about 5×10⁶-1×10⁹, about 5×10⁶-3×10⁸, about 5×10⁶-1×10⁸, 5×10⁶-3×10⁷, or about 5×10⁶-1×10⁷ mononuclear cells (e.g., PBMCs such as G-CSF mobilized PBMCs). In some embodiments, a population of human mononuclear cells comprises about 5×10⁶ to about 1×10⁷ mononuclear cells (e.g., PBMCs such as G-CSF mobilized PBMCs).

In some embodiments, a population of human mononuclear cells, human polymorphonuclear leukocytes, or both is a population of human polymorphonuclear leukocytes. Polymorphonuclear leukocytes (PMNs) are a type of white blood cell and include, e.g., neutrophils, eosinophils, basophils, and mast cells. In some embodiments, a population of human polymorphonuclear leukocytes comprises peripheral blood polymorphonuclear leukocytes, umbilical cord blood polymorphonuclear leukocytes, bone marrow polymorphonuclear leukocytes, or a combination thereof.

In some embodiments, a population of human polymorphonuclear leukocytes comprises about 5×10² to about 1×10⁹ polymorphonuclear leukocytes, for example, about 5×10²-3×10⁸, about 5×10²-1×10⁸, about 5×10²-3×10⁷, about 5×10²-1×10⁷, about 5×10³-1×10⁹, about 5×10³-3×10⁸, about 5×10³-1×10⁸, about 5×10³-3×10⁷, about 5×10³-1×10⁷, about 5×10⁴-1×10⁹, about 5×10⁴-3×10⁸, about 5×10⁴-1×10⁸, about 5×10⁴-3×10⁷, about 5×10⁴-1×10⁷, about 5×10⁵-1×10⁹, about 5×10⁵-3×10⁸, about 5×10⁵-1×10⁸, about 5×10⁵-3×10⁷, about 5×10⁵-1×10⁷, about 5×10⁶-1×10⁹, about 5×10⁶-3×10⁸, about 5×10⁶-1×10⁸, about 5×10⁶-3×10⁷ or about 5×10⁶-1×10⁷ polymorphonuclear leukocytes. In some embodiments, a population of human polymorphonuclear leukocytes comprises about 5×10⁶ to about 1×10⁷ polymorphonuclear leukocytes.

In some embodiments, a population of human mononuclear cells, human polymorphonuclear leukocytes, or both is a population of human mononuclear cells and human polymorphonuclear leukocytes. In some embodiments, a population of human mononuclear cells and human polymorphonuclear leukocytes comprises about 5×10² to about 1×10⁹ mononuclear cells and polymorphonuclear leukocytes, for example, about 5×10²-3×10⁸, about 5×10²-1×10⁸, about 5×10²-3×10⁷, about 5×10²-1×10⁷, about 5×10³-1×10⁹, about 5×10³-3×10⁸, about 5×10³-1×10⁸, about 5×10³-3×10⁷, about 5×10³-1×10⁷, about 5×10⁴-1×10⁹, about 5×10⁴-3×10⁸, about 5×10⁴-1×10⁸, about 5×10⁴-3×10⁷, about 5×10⁴-1×10⁷, about 5×10⁵-1×10⁹, about 5×10⁵-3×10⁸, about 5×10⁵-1×10⁸, about 5×10⁵-3×10⁷, about 5×10⁵-1×10⁷, about 5×10⁶-1×10⁹, about 5×10⁶-3×10⁸, about 5×10⁶-1×10⁸, about 5×10⁶-3×10⁷ or about 5×10⁶-1×10⁷ mononuclear cells and polymorphonuclear leukocytes.

In some embodiments, a method disclosed herein comprises engrafting a population of human mononuclear cells, human polymorphonuclear leukocytes, or both disclosed herein from a human (e.g., healthy human donor) into a non-human animal (e.g., a mouse, such as a NRGS or NSGS mouse) lacking endogenous mature T cells. In some embodiments, a method comprises engrafting a population of human mononuclear cells from a human into a non-human animal lacking endogenous mature T cells. In some embodiments, a method comprises engrafting a population of human polymorphonuclear leukocytes disclosed herein from a human into a non-human animal lacking endogenous mature T cells. In some embodiments, a method comprises engrafting a population of human mononuclear cells and human polymorphonuclear leukocytes disclosed herein from a human into a non-human animal lacking endogenous mature T cells.

In some embodiments, a population of human mononuclear cells, human polymorphonuclear leukocytes, or both comprises about 0-5% T cells. In some embodiments, a population of human mononuclear cells, human polymorphonuclear leukocytes, or both is T-cell depleted (e.g., comprising 0% T cells). In some embodiments, a method disclosed herein further comprises depleting T cells from a population of human mononuclear cells, human polymorphonuclear leukocytes, or both, e.g., prior to engrafting the population of human mononuclear cells, human polymorphonuclear leukocytes, or both, into a non-human animal. In some embodiments, a population of human mononuclear cells, human polymorphonuclear leukocytes, or both is not T-cell depleted.

In some embodiments, a population of human mononuclear cells (e.g., PBMCs such as G-CSF mobilized PBMCs) comprises about 0-5% T cells. In some embodiments, a population of human mononuclear cells (e.g., PBMCs such as G-CSF mobilized PBMCs) is T-cell depleted (e.g., comprising 0% T cells). In some embodiments, a method disclosed herein comprises depleting T cells from a population of human mononuclear cells. In some embodiments, a population of human mononuclear cells is not T-cell depleted.

In some embodiments, a population of human polymorphonuclear leukocytes comprises about 0-5% T cells. In some embodiments, a population of human polymorphonuclear leukocytes is T-cell depleted (e.g., comprising 0% T cells). In some embodiments, a method disclosed herein comprises depleting T cells from a population of human polymorphonuclear leukocytes. In some embodiments, a population of human polymorphonuclear leukocytes is not T-cell depleted.

In some embodiments, a population of human mononuclear cells and human polymorphonuclear leukocytes comprises about 0-5% T cells. In some embodiments, a population of human mononuclear cells and human polymorphonuclear leukocytes is T-cell depleted (e.g., comprising 0% T cells). In some embodiments, a method disclosed herein comprises depleting T cells from a population of human mononuclear cells and human polymorphonuclear leukocytes. In some embodiments, a population of human mononuclear cells and human polymorphonuclear leukocytes is not T-cell depleted.

In some embodiments, a population of human mononuclear cells, human polymorphonuclear leukocytes, or both further comprises a population of human B-cells (e.g., CD19⁺ B cells) as, for example, when the population of human mononuclear cells, human polymorphonuclear leukocytes, or both comprises human PBMCs. In some embodiments, a population of human mononuclear cells, human polymorphonuclear leukocytes, or both comprises a population of human B-cells (e.g., CD19⁺ B cells) and about 0-5% human T cells (e.g., is T-cell depleted).

### Human Lymphocytes

In some embodiments, a method disclosed herein comprises a single engraftment, e.g., a single engraftment of a population of human mononuclear cells, human polymorphonuclear leukocytes, or both (such as from one or more healthy humans) into a non-human animal (such as a mouse, such as a NRGS mouse or a NSGS mouse).

In some embodiments, a method disclosed herein comprises at least two engraftments (e.g., two engraftments, three engraftments, four engraftments, etc.) e.g., at least two engraftments (such as 2, 3, 4, 5, 6, 7, 8, 9, or 10 engraftments) of a population of human mononuclear cells, human polymorphonuclear leukocytes, or both (such as from one or more healthy humans) into a non-human animal (such as a mouse, such as a NRGS mouse or a NSGS mouse). In some embodiments, a method disclosed herein comprises engrafting a population of human lymphocytes (such as from one or more healthy humans) into a non-human animal after engrafting a population of human mononuclear cells, human polymorphonuclear leukocytes, or both (such as from one or more healthy humans, such as from the same one or more healthy humans as the aforementioned human lymphocytes) into the non-human animal. In some embodiments, a method disclosed herein comprises engrafting a population of human lymphocytes into a non-human animal after engrafting a population of human mononuclear cells from the human into the non-human animal. In some embodiments, a method disclosed herein comprises engrafting a population of human lymphocytes into a non-human animal after engrafting a population of human polymorphonuclear leukocytes into the non-human animal. In some embodiments, a method disclosed herein comprises engrafting a population of human lymphocytes from a human into a non-human animal after engrafting a population of human mononuclear cells and human polymorphonuclear leukocytes into the non-human animal. In some embodiments, a functional human immune system is generated from an engraftment of a population of human mononuclear cells, human polymorphonuclear leukocytes, or both, and an engraftment of a population of human lymphocytes (e.g., from a healthy human donor) thereafter. In some embodiments, the human lymphocytes, human monocytes, and human polymorphonuclear cells are from the same human. In some embodiments, the human lymphocytes and human monocytes are from the same human. In some embodiments, the human lymphocytes and human polymorphonuclear cells are from the same human.

In some embodiments, a population of human lymphocytes is engrafted after engrafting a population of human mononuclear cells, human polymorphonuclear leukocytes, or both into the non-human animal. For example, in some embodiments, a population of human lymphocytes is engrafted about 5-29 days after engrafting a population of human mononuclear cells, human polymorphonuclear leukocytes, or both into the non-human animal, for example, about: 5, 6, 7, 8, 9, 10, 11, 12, 13, 14, 15, 16, 17, 18, 19, 20, 21, 22, 23, 24, 25, 26, 27, 28, 29, 6-8, or 27-29 days after engrafting a population of human mononuclear cells, human polymorphonuclear leukocytes, or both into a non-human animal. In some embodiments, a population of human lymphocytes is engrafted about 27-29 days (e.g., about 28 days) after engrafting a population of human mononuclear cells, human polymorphonuclear leukocytes, or both into a non-human animal. In some embodiments, a population of human lymphocytes is engrafted about 6-8 days (e.g., about 7 days) after engrafting a population of human mononuclear cells, human polymorphonuclear leukocytes, or both into a non-human animal.

In some embodiments, a population of human lymphocytes comprises total T cells. In some embodiments, a population of human lymphocytes comprises about 1×10²-5×10⁹ total T cells, for example, about: 1×10²-1×10⁹, 1×10²-3×10⁸, 1×10²-1×10⁸, 1×10²-3×10⁷, 1×10²-1×10⁷, 1×10²-3×10⁶, 1×10²-1×10⁶, 3×10²-5×10⁹, 3×10²-1×10⁹, 3×10²-3×10⁸, 3×10²-1×10⁸, 3×10²-3×10⁷, 3×10²-1×10⁷, 3×10²-3×10⁶, 3×10²-1×10⁶, 1×10³-5×10⁹, 1×10³-1×10⁹, 1×10³-3×10⁸, 1×10³-1×10⁸, 1×10³-3×10⁷, 1×10³-1×10⁷, 1×10³-3×10⁶, 1×10³-1×10⁶, 3×10³-5×10⁹, 3×10³-1×10⁹, 3×10³-3×10⁸, 3×10³-1×10⁸, 3×10³-3×10⁷, 3×10³-1×10⁷, 3×10³-3×10⁶, 3×10³-1×10⁶, 1×10⁴-5×10⁹, 1×10⁴-1×10⁹, 1×10⁴-3×10⁸, 1×10⁴-1×10⁸, 1×10⁴-3×10⁷, 1×10⁴-1×10⁷, 1×10⁴-3×10⁶, 1×10⁴-1×10⁶, 3×10⁴-5×10⁹, 3×10⁴-1×10⁹, 3×10⁴-3×10⁸, 3×10⁴-1×10⁸, 3×10⁴-3×10⁷, 3×10⁴-1×10⁷, 3×10⁴-3×10⁶, 3×10⁴-1×10⁶, 1×10⁵-5×10⁹, 1×10⁵-1×10⁹, 1×10⁵-3×10⁸, 1×10⁵-1×10⁸, 1×10⁵-3×10⁷, 1×10⁵-1×10⁷, 1×10⁵-3×10⁶, 1×10⁵-1×10⁶, 3×10⁵-5×10⁹, 3×10⁵-1×10⁹, 3×10⁵-3×10⁸, 3×10⁵-1×10⁸, 3×10⁵-3×10⁷, 3×10⁵-1×10⁷, 3×10⁵-3×10⁶, 5×10⁵-2×10⁶, 3×10⁵-1×10⁶, 1×10⁶-5×10⁹, 1×10⁶-1×10⁹, 1×10⁶-3×10⁸, 1×10⁶-1×10⁸, 1×10⁶-3×10⁷, 1×10⁶-1×10⁷ or 1×10⁶-3×10⁶ total T cells. In some embodiments, a population of human lymphocytes comprises about 5×10⁵-2×10⁶ total T cells. In some embodiments, a population of human lymphocytes comprises about 1×10⁶ total T cells.

In some embodiments, a population of human lymphocytes comprises CD4⁺ T cells. In some embodiments, a population of human lymphocytes comprises about 1×10²-5×10⁹ CD4⁺ T cells, for example, about: 1×10²-1×10⁹, 1×10²-3×10⁸, 1×10²-1×10⁸, 1×10²-3×10⁷, 1×10²-1×10⁷, 1×10²-3×10⁶, 1×10²-1×10⁶, 3×10²-5×10⁹, 3×10²-1×10⁹, 3×10²-3×10⁸, 3×10²-1×10⁸, 3×10²-3×10⁷, 3×10²-1×10⁷, 3×10²-3×10⁶, 3×10²-1×10⁶, 1×10³-5×10⁹, 1×10³-1×10⁹, 1×10³-3×10⁸, 1×10³-1×10⁸, 1×10³-3×10⁷, 1×10³-1×10⁷, 1×10³-3×10⁶, 1×10³-1×10⁶, 3×10³-5×10⁹, 3×10³-1×10⁹, 3×10³-3×10⁸, 3×10³-1×10⁸, 3×10³-3×10⁷, 3×10³-1×10⁷, 3×10³-3×10⁶, 3×10³-1×10⁶, 1×10⁴-5×10⁹, 1×10⁴-1×10⁹, 1×10⁴-3×10⁸, 1×10⁴-1×10⁸, 1×10⁴-3×10⁷, 1×10⁴-1×10⁷, 1×10⁴-3×10⁶, 1×10⁴-1×10⁶, 3×10⁴-5×10⁹, 3×10⁴-1×10⁹, 3×10⁴-3×10⁸, 3×10⁴-1×10⁸, 3×10⁴-3×10⁷, 3×10⁴-1×10⁷, 3×10⁴-3×10⁶, 3×10⁴-1×10⁶, 1×10⁵-5×10⁹, 1×10⁵-1×10⁹, 1×10⁵-3×10⁸, 1×10⁵-1×10⁸, 1×10⁵-3×10⁷, 1×10⁵-1×10⁷, 1×10⁵-3×10⁶, 1×10⁵-1×10⁶, 3×10⁵-5×10⁹, 3×10⁵-1×10⁹, 3×10⁵-3×10⁸, 3×10⁵-1×10⁸, 3×10⁵-3×10⁷, 3×10⁵-1×10⁷, 3×10⁵-3×10⁶, 5×10⁵-2×10⁶, 3×10⁵-1×10⁶, 1×10⁶-5×10⁹, 1×10⁶-1×10⁹, 1×10⁶-3×10⁸, 1×10⁶-1×10⁸, 1×10⁶-3×10⁷, 1×10⁶-1×10⁷ or 1×10⁶-3×10⁶ CD4⁺ T cells. In some embodiments, the population of human lymphocytes comprises about 5×10⁵-2×10⁶ CD4⁺ T cells. In some embodiments, the population of human lymphocytes comprises about 1×10⁶ CD4⁺ T cells.

In some embodiments, a population of human CD4⁺ T cells is engrafted after engrafting a population of human mononuclear cells, human polymorphonuclear leukocytes, or both into the non-human animal. In some embodiments, a population of human CD4⁺ T cells is engrafted after engrafting a population of human mononuclear cells into the non-human animal. In some embodiments, a population of human CD4⁺ T cells is engrafted after engrafting a population of human polymorphonuclear leukocytes into the non-human animal. In some embodiments, a population of human CD4⁺ T cells is engrafted after engrafting a population of human mononuclear cells and human polymorphonuclear leukocytes into the non-human animal. For example, in some embodiments, a population of human CD4⁺ T cells is engrafted at least about 21 days after engrafting a population of human mononuclear cells, human polymorphonuclear leukocytes, or both into the non-human animal, for example, at least about: 22, 23, 24, 25, 26, 27, 28, 29 or 30 days after engrafting a population of human mononuclear cells, human polymorphonuclear leukocytes, or both into a non-human animal. In some embodiments, a population of CD4⁺ T cells is engrafted about: 21, 22, 23, 24, 25, 26, 27, 28, 29 or 30 days after engrafting a population of human mononuclear cells, human polymorphonuclear leukocytes, or both into a non-human animal. In some embodiments, a population of CD4⁺ T cells is engrafted about 27-29 days (e.g., about 28 days) after engrafting a population of human mononuclear cells, human polymorphonuclear leukocytes, or both into a non-human animal.

In some embodiments, a population of human lymphocytes comprises CD8⁺ T cells. In some embodiments, the population of human lymphocytes comprises about 1×10²-5×10⁹ CD8⁺ T cells, for example, about: 1×10²-1×10⁹, 1×10²-3×10⁸, 1×10²-1×10⁸, 1×10²-3×10⁷, 1×10²-1×10⁷, 1×10²-3×10⁶, 1×10²-1×10⁶, 3×10²-5×10⁹, 3×10²-1×10⁹, 3×10²-3×10⁸, 3×10²-1×10⁸, 3×10²-3×10⁷, 3×10²-1×10⁷, 3×10²-3×10⁶, 3×10²-1×10⁶, 1×10³-5×10⁹, 1×10³-1×10⁹, 1×10³-3×10⁸, 1×10³-1×10⁸, 1×10³-3×10⁷, 1×10³-1×10⁷, 1×10³-3×10⁶, 1×10³-1×10⁶, 3×10³-5×10⁹, 3×10³-1×10⁹, 3×10³-3×10⁸, 3×10³-1×10⁸, 3×10³-3×10⁷, 3×10³-1×10⁷, 3×10³-3×10⁶, 3×10³-1×10⁶, 1×10⁴-5×10⁹, 1×10⁴-1×10⁹, 1×10⁴-3×10⁸, 1×10⁴-1×10⁸, 1×10⁴-3×10⁷, 1×10⁴-1×10⁷, 1×10⁴-3×10⁶, 1×10⁴-1×10⁶, 3×10⁴-5×10⁹, 3×10⁴-1×10⁹, 3×10⁴-3×10⁸, 3×10⁴-1×10⁸, 3×10⁴-3×10⁷, 3×10⁴-1×10⁷, 3×10⁴-3×10⁶, 3×10⁴-1×10⁶, 1×10⁵-5×10⁹, 1×10⁵-1×10⁹, 1×10⁵-3×10⁸, 1×10⁵-1×10⁸, 1×10⁵-3×10⁷, 1×10⁵-1×10⁷, 1×10⁵-3×10⁶, 1×10⁵-1×10⁶, 3×10⁵-5×10⁹, 3×10⁵-1×10⁹, 3×10⁵-3×10⁸, 3×10⁵-1×10⁸, 3×10⁵-3×10⁷, 3×10⁵-1×10⁷, 3×10⁵-3×10⁶, 5×10⁵-2×10⁶, 3×10⁵-1×10⁶, 1×10⁶-5×10⁹, 1×10⁶-1×10⁹, 1×10⁶-3×10⁸, 1×10⁶-1×10⁸, 1×10⁶-3×10⁷, 1×10⁶-1×10⁷ or 1×10⁶-3×10⁶ CD8⁺ T cells. In some embodiments, the population of human lymphocytes comprises about 5×10⁵-2×10⁶ CD8⁺ T cells. In some embodiments, the population of human lymphocytes comprises about 1×10⁶ CD8⁺ T cells.

In some embodiments, a population of human CD8⁺ T cells is engrafted after engrafting a population of human mononuclear cells, human polymorphonuclear leukocytes, or both into the non-human animal. In some embodiments, a population of human CD8⁺ T cells is engrafted after engrafting a population of human mononuclear cells into the non-human animal. In some embodiments, a population of human CD8⁺ T cells is engrafted after engrafting a population of human polymorphonuclear leukocytes into the non-human animal. In some embodiments, a population of human CD8⁺ T cells is engrafted after engrafting a population of human mononuclear cells and human polymorphonuclear leukocytes into the non-human animal. For example, in some embodiments, a population of human CD8⁺ T cells is engrafted at least about 14 days after engrafting a population of human mononuclear cells, human polymorphonuclear leukocytes, or both into the non-human animal, for example, at least about: 15, 16, 17, 18, 19, 20, 21, 22, 23, 24, 25, 26, 27, 28, 29 or 30 days after engrafting the population of human mononuclear cells, human polymorphonuclear leukocytes, or both into a non-human animal. In some embodiments, a population of human CD8⁺ T cells is engrafted about: 14, 15, 16, 17, 18, 19, 20, 21, 22, 23, 24, 25, 26, 27, 28, 29 or 30 days after engrafting a population of human mononuclear cells, human polymorphonuclear leukocytes, or both into a non-human animal. In some embodiments, a population of human CD8⁺ T cells is engrafted about 27-29 days (e.g., about 28 days) after engrafting a population of human mononuclear cells, human polymorphonuclear leukocytes, or both into a non-human animal.

In some embodiments, a population of human lymphocytes comprises CD4⁺ T cells and CD8⁺ T cells. In some embodiments, the population of human lymphocytes comprises about 1×10²-5×10⁹ CD4⁺ T cells and CD8⁺ T cells, for example, about: 1×10²-1×10⁹, 1×10²-3×10⁸, 1×10²-1×10⁸, 1×10²-3×10⁷, 1×10²-1×10⁷, 1×10²-3×10⁶, 1×10²-1×10⁶, 3×10²-5×10⁹, 3×10²-1×10⁹, 3×10²-3×10⁸, 3×10²-1×10⁸, 3×10²-3×10⁷, 3×10²-1×10⁷, 3×10²-3×10⁶, 3×10²-1×10⁶, 1×10³-5×10⁹, 1×10³-1×10⁹, 1×10³-3×10⁸, 1×10³-1×10⁸, 1×10³-3×10⁷, 1×10³-1×10⁷, 1×10³-3×10⁶, 1×10³-1×10⁶, 3×10³-5×10⁹, 3×10³-1×10⁹, 3×10³-3×10⁸, 3×10³-1×10⁸, 3×10³-3×10⁷, 3×10³-1×10⁷, 3×10³-3×10⁶, 3×10³-1×10⁶, 1×10⁴-5×10⁹, 1×10⁴-1×10⁹, 1×10⁴-3×10⁸, 1×10⁴-1×10⁸, 1×10⁴-3×10⁷, 1×10⁴-1×10⁷, 1×10⁴-3×10⁶, 1×10⁴-1×10⁶, 3×10⁴-5×10⁹, 3×10⁴-1×10⁹, 3×10⁴-3×10⁸, 3×10⁴-1×10⁸, 3×10⁴-3×10⁷, 3×10⁴-1×10⁷, 3×10⁴-3×10⁶, 3×10⁴-1×10⁶, 1×10⁵-5×10⁹, 1×10⁵-1×10⁹, 1×10⁵-3×10⁸, 1×10⁵-1×10⁸, 1×10⁵-3×10⁷, 1×10⁵-1×10⁷, 1×10⁵-3×10⁶, 1×10⁵-1×10⁶, 3×10⁵-5×10⁹, 3×10⁵-1×10⁹, 3×10⁵-3×10⁸, 3×10⁵-1×10⁸, 3×10⁵-3×10⁷, 3×10⁵-1×10⁷, 3×10⁵-3×10⁶, 5×10⁵-2×10⁶, 3×10⁵-1×10⁶, 1×10⁶-5×10⁹, 1×10⁶-1×10⁹, 1×10⁶-3×10⁸, 1×10⁶-1×10⁸, 1×10⁶-3×10⁷, 1×10⁶-1×10⁷ or 1×10⁶-3×10⁶ CD4⁺ T cells and CD8⁺ T cells. In some embodiments, the population of human lymphocytes comprises about 5×10⁵-2×10⁶ CD4⁺ T cells and CD8⁺ T cells. In some embodiments, the population of human lymphocytes comprises about 1×10⁶ CD4⁺ T cells and CD8⁺ T cells.

In some embodiments, a population of human lymphocytes comprises a population of B cells. In some embodiments, a population of B cells comprises naïve B cells, memory B cells, plasma cells, or a combination thereof. In some embodiments, a population of human lymphocytes comprises naïve B cells. In some embodiments, the population of human lymphocytes comprises memory B cells. In some embodiments, the population of B cells comprises plasma cells. In some embodiments, a population of B cells comprises of naïve B cells, memory B cells, and plasma cells. In some embodiments, a population of B cells comprises CD19⁺ B cells (e.g., CD19⁺ naïve B cells).

In some embodiments, the population of human lymphocytes comprises about 1×10²-1×10⁹ B cells (e.g., CD19⁺ B cells), for example, about: 1×10²-3×10⁸, 1×10²-1×10⁸, 1×10²-3×10⁷, 1×10²-1×10⁷, 1×10²-3×10⁶, 1×10²-1×10⁶, 1×10²-3×10⁵, 3×10²-1×10⁹, 3×10²-3×10⁸, 3×10²-1×10⁸, 3×10²-3×10⁷, 3×10²-1×10⁷, 3×10²-3×10⁶, 3×10²-1×10⁶, 3×10²-3×10⁵, 1×10³-1×10⁹, 1×10³-3×10⁸, 1×10³-1×10⁸, 1×10³-3×10⁷, 1×10³-1×10⁷, 1×10³-3×10⁶, 1×10³-1×10⁶, 1×10³-3×10⁵, 3×10³-1×10⁹, 3×10³-3×10⁸, 3×10³-1×10⁸, 3×10³-3×10⁷, 3×10³-1×10⁷, 3×10³-3×10⁶, 3×10³-1×10⁶, 3×10³-3×10⁵, 1×10⁴-1×10⁹, 1×10⁴-3×10⁸, 1×10⁴-1×10⁸, 1×10⁴-3×10⁷, 1×10⁴-1×10⁷, 1×10⁴-3×10⁶, 1×10⁴-1×10⁶, 1×10⁴-3×10⁵, 3×10⁴-1×10⁹, 3×10⁴-3×10⁸, 3×10⁴-1×10⁸, 3×10⁴-3×10⁷, 3×10⁴-1×10⁷, 3×10⁴-3×10⁶, 3×10⁴-1×10⁶, 3×10⁴-3×10⁵, 1×10⁵-1×10⁹, 1×10⁵-3×10⁸, 1×10⁵-1×10⁸, 1×10⁵-3×10⁷, 1×10⁵-1×10⁷, 1×10⁵-3×10⁶, 1×10⁵-1×10⁶, 1×10⁵-3×10⁵, 2.5×10⁵-1×10⁶, 3×10⁵-1×10⁹, 3×10⁵-3×10⁸, 3×10⁵-1×10⁸, 3×10⁵-3×10⁷, 3×10⁵-1×10⁷, 3×10⁵-3×10⁶ or 3×10⁵-1×10⁶ B cells (e.g., CD19⁺ B cells). In some embodiments, a population of human lymphocytes comprises about 2.5×10⁵-1×10⁶ B cells (e.g., CD19⁺ B cells). In some embodiments, a population of human lymphocytes comprises about 5×10⁵ B cells (e.g., CD19⁺ B cells).

In some embodiments, a population of human CD19⁺ B cells is engrafted after engrafting a population of human mononuclear cells, human polymorphonuclear leukocytes, or both into the non-human animal. In some embodiments, a population of human CD19⁺ B cells is engrafted after engrafting a population of human mononuclear cells into the non-human animal. In some embodiments, a population of human CD19⁺ B cells is engrafted after engrafting a population of human polymorphonuclear leukocytes into the non-human animal. In some embodiments, a population of human CD19⁺ B cells is engrafted after engrafting a population of human mononuclear cells and human polymorphonuclear leukocytes into the non-human animal. For example, in some embodiments, a population of human CD19⁺ B cells is engrafted at least about 21 days after engrafting a population of human mononuclear cells, human polymorphonuclear leukocytes, or both into the non-human animal, for example, at least about: 22, 23, 24, 25, 26, 27, 28, 29 or 30 days after engrafting a population of human mononuclear cells, human polymorphonuclear leukocytes, or both into a non-human animal. In some embodiments, a population of human CD19⁺ B cells is engrafted about: 21, 22, 23, 24, 25, 26, 27, 28, 29 or 30 days after engrafting a population of human mononuclear cells, human polymorphonuclear leukocytes, or both into a non-human animal. In some embodiments, a population of human CD19⁺ B cells is engrafted about 27-29 days (e.g., about 28 days) after engrafting a population of human mononuclear cells, human polymorphonuclear leukocytes, or both into a non-human animal.

In some embodiments, a population of human mononuclear cells, human polymorphonuclear leukocytes, or both (e.g., a population of T-cell depleted PBMC comprising CD19⁺ B cells) is engrafted, then a population of human CD4⁺ T cells is engrafted, and then a population of human CD8⁺ T cells is engrafted.

### TIM Receptor Agonists

It is believed that T cell immunoglobulin and mucin domain (TIM) receptor agonists can also be used to prepare a non-human animal described herein for engraftment. Thus, in some embodiments, a non-human animal (e.g., a mouse, such as a NRGS or NSGS mouse) is administered a TIM receptor agonist. TIM receptors are type 1 cell-surface glycoproteins, and TIM1, TIM3 and TIM4, TIM receptors expressed in humans, have been identified as phosphatidylserine receptors. TIM1 is preferentially expressed on T-helper 2 cells, and operates as a potent costimulatory molecule for T-cell activation. TIM3 is preferentially expressed on T-helper 1 cells, type 1 T-cells and dendritic cells, and generates an inhibitory signal resulting in apoptosis of T-helper 1 cells and type 1 T-cells. TIM4 is expressed on antigen-presenting cells, and mediates phagocytosis of apoptotic cells, thereby promoting tolerance. In some embodiments, a TIM receptor is a TIM3 receptor. In some embodiments, a TIM receptor is a TIM4 receptor. In some embodiments, a TIM receptor is a TIM1 receptor. “TIM” is also referred to, for example, in the literature, as “Tim.”

Examples of TIM receptor agonists are disclosed in International Patent Application Nos. PCT/US2022/071082 (International Publication No. WO 2022/192899), PCT/US2022/74903 (International Publication No. WO 2023/019242), and PCT/US2022/74908 (International Publication No. WO 2023/019244), the entire contents of which are incorporated herein by reference. In some embodiments, a non-human animal (e.g., a NRGS or NSGS mouse) is administered a compound, or a pharmaceutically acceptable salt thereof, or composition of International Patent Application Nos. PCT/US2022/071082 (International Publication No. WO 2022/192899), PCT/US2022/74903 (International Publication No. WO 2023/019242), and/or PCT/US2022/74908 (International Publication No. WO 2023/019244) (e.g., in an amount sufficient to prepare the non-human animal to an engraftment and/or to inhibit rejection of the engraftment). In some embodiments, a method disclosed herein further comprises administering a compound, or a pharmaceutically acceptable salt thereof, or a composition of International Patent Application No. PCT/US2022/071082 (International Publication No. WO 2022/192899), PCT/US2022/74903 (International Publication No. WO 2023/019242), and/or PCT/US2022/74908 (International Publication No. WO 2023/019244), to a non-human animal disclosed herein (e.g., a mouse, such as a NRGS or NSGS mouse) (e.g., in an amount sufficient to prepare the non-human animal to an engraftment and/or to inhibit rejection of the engraftment). Other TIM receptor agonists include those disclosed in U.S. Patent Application Publication Nos. US 2016/0243220 and US 2019/0151426, the entire contents of which are incorporated by reference herein, such as O-phospho-L-serine, phosphatidylserine and lyso-phosphatidylserine. In some embodiments, a non-human animal (e.g., a mouse, such as a NRGS or NSGS mouse) is administered a compound, or a pharmaceutically acceptable salt thereof, or composition of U.S. Patent Application Publication Nos. US 2016/0243220 and/or US 2019/0151426 (e.g., in an amount sufficient to prepare the non-human animal to an engraftment and/or to inhibit rejection of the engraftment). In some embodiments, a method disclosed herein further comprises administering a compound, or a pharmaceutically acceptable salt thereof, or a composition of U.S. Patent Application Publication Nos. US 2016/0243220 and/or US 2019/0151426, to a non-human animal disclosed herein (e.g., a mouse, such as a NRGS or NSGS mouse) (e.g., in an amount sufficient to prepare the non-human animal to an engraftment and/or to inhibit rejection of the engraftment).

In some embodiments, a method disclosed herein further comprises administering a TIM receptor agonist to the non-human animal, e.g., in an amount sufficient to prepare the non-human animal for engraftment (e.g., of T cells) and/or to inhibit rejection of the engraftment. The TIM receptor agonist can be administered prior to (such as daily for five days prior to), concurrently with, or after an engraftment.

In some embodiments, a population of human mononuclear cells, human polymorphonuclear leukocytes, or both is incubated with a TIM receptor agonist, such as any TIM receptor agonist described herein. In some embodiments, a method disclosed herein comprises incubating a population of human mononuclear cells, human polymorphonuclear leukocytes, or both with a TIM receptor agonist, e.g., for about five days, before engrafting the population into a non-human animal.

In some embodiments, a population of human mononuclear cells is incubated with a TIM receptor agonist, such as any TIM receptor agonist described herein. In some embodiments, a method disclosed herein comprises incubating a population of human mononuclear cells with a TIM receptor agonist, e.g., for about five days, before engrafting the population into a non-human animal.

In some embodiments, a population of human polymorphonuclear leukocytes is incubated with a TIM receptor agonist, such as any TIM receptor agonist described herein. In some embodiments, a method disclosed herein comprises incubating a population of human polymorphonuclear leukocytes with a TIM receptor agonist, e.g., for about five days, before engrafting the population into a non-human animal.

In some embodiments, a population of human mononuclear cells and human polymorphonuclear leukocytes is incubated with a TIM receptor agonist, such as any TIM receptor agonist described herein. In some embodiments, a method disclosed herein comprises incubating a population of human mononuclear cells and human polymorphonuclear leukocytes with a TIM receptor agonist, e.g., for about 5 days, before engrafting the population into a non-human animal.

In some embodiments, a population of human lymphocytes (e.g., CD4⁺ T cells and/or CD19⁺ B cells) is incubated with a TIM receptor agonist, such as any TIM receptor agonist described herein (e.g., for about five days) before engrafting into a non-human animal, and is engrafted (e.g., about seven days) after engrafting a population of human mononuclear cells, human polymorphonuclear leukocytes, or both into a non-human animal.

In some embodiments, a method disclosed herein comprises incubating a population of human lymphocytes (e.g., CD4⁺ T cells and/or CD19⁺ B cells) with a TIM receptor agonist, such as any TIM receptor agonist described herein (e.g., for about 5 days) before engrafting into a non-human animal, and engrafting the population of human lymphocytes into a non-human animal (e.g., about 7 days) after engrafting a population of human mononuclear cells, human polymorphonuclear leukocytes, or both into the non-human animal.

### Lacking Cancer Cells

In some embodiments, a population of human mononuclear cells, human polymorphonuclear leukocytes, or both does not comprise cancer cells (e.g., hematological cancer cells). In some embodiments, a population of human lymphocytes does not comprise cancer cells (e.g., hematological cancer cells). In some embodiments, a population of human mononuclear cells, human polymorphonuclear leukocytes, or both and a population of human lymphocytes do not comprise cancer cells (e.g., hematological cancer cells). In some embodiments, the cancer cells are human cancer cells.

In some embodiments, a non-human animal described herein lacks human cancer cells (e.g., human hematological cancer cells).

In some embodiments, cancer cells may be of a hematological malignancy, “hematological cancer cells.” Examples of hematological malignancies include leukemia, such as acute lymphocytic leukemia (ALL), chronic lymphocytic leukemia (CLL), acute myeloid leukemia (AML) or chronic myeloid leukemia (CML), myeloma, and lymphoma. In other embodiments, cancer cells may be of a solid tumor, “solid tumor cancer cells.” Examples of solid tumors include tumors of the breast, lung, prostate, colon, bladder, ovary, kidney, stomach, colon, rectum, testes, head and/or neck, pancreas, brain and skin.

Other non-limiting examples of cancers include acute lymphoblastic leukemia (ALL); acute myeloid leukemia (AML); adrenocortical carcinoma; adrenocortical carcinoma, childhood; Acquired immunodeficiency syndrome (AIDS)-related cancer (e.g., Kaposi Sarcoma, AIDS-related lymphoma, primary central nervous system (CNS) lymphoma); anal cancer; appendix cancer; astrocytoma, childhood; atypical teratoid/rhabdoid tumor, childhood, CNS; basal cell carcinoma of the skin; bile duct cancer; bladder cancer; bladder cancer, childhood; bone cancer (including Ewing sarcoma, osteosarcoma and malignant fibrous histiocytoma); brain tumors/cancer; breast cancer; Burkitt lymphoma; carcinoid tumor (gastrointestinal); carcinoid tumor, childhood; cardiac (heart) tumors, childhood; embryonal tumors, childhood; germ cell tumor, childhood; primary CNS lymphoma; cervical cancer; childhood cervical cancer; cholangiocarcinoma; chordoma, childhood; chronic lymphocytic leukemia (CLL); chronic myelogenous leukemia (CML); chronic myeloproliferative neoplasms; colorectal cancer; childhood colorectal cancer; craniopharyngioma, childhood; cutaneous T-cell lymphoma (e.g., mycosis fungoides and Sézary syndrome); ductal carcinoma in situ (DCIS); embryonal tumors, central nervous system, childhood; endometrial cancer (uterine cancer); ependymoma, childhood; esophageal cancer; childhood esophageal cancer; esthesioneuroblastoma; Ewing sarcoma; extracranial germ cell tumor, childhood; extragonadal germ cell tumor; eye (ocular) cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST); childhood gastrointestinal stromal tumors; germ cell tumors; childhood CNS germ cell tumors (e.g., childhood extracranial germ cell tumors, extragonadal germ cell tumors, ovarian germ cell tumors, testicular cancer); gestational trophoblastic disease; hairy cell leukemia; head and neck cancer; heart tumors, childhood; hepatocellular (liver) cancer; histiocytosis, Langerhans cell; Hodgkin lymphoma; hypopharyngeal cancer; intraocular melanoma; childhood intraocular melanoma; islet cell tumors, pancreatic neuroendocrine tumors; Kaposi sarcoma; kidney (renal cell) cancer; Langerhans cell histiocytosis; laryngeal cancer; leukemia; lip and oral cavity cancer; liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; Merkel cell carcinoma; mesothelioma, malignant; childhood mesothelioma; metastatic cancer; metastatic squamous neck cancer with occult primary; midline tract carcinoma with NUT gene changes; mouth cancer; multiple endocrine neoplasia syndromes; multiple myeloma/plasma cell neoplasms; mycosis fungoides; myelodysplastic syndromes, myelodysplastic/myeloproliferative neoplasms; myelogenous leukemia, chronic (CML); myeloid leukemia, acute (AML); myeloproliferative neoplasms, chronic; nasal cavity and paranasal sinus cancer; nasopharyngeal cancer; neuroblastoma; non-Hodgkin lymphoma; non-small cell lung cancer; oral cancer, lip and oral cavity cancer and oropharyngeal cancer; osteosarcoma and malignant fibrous histiocytoma of bone; ovarian cancer; childhood ovarian cancer; pancreatic cancer; childhood pancreatic cancer; pancreatic neuroendocrine tumors; papillomatosis (childhood laryngeal); paraganglioma; childhood paraganglioma; paranasal sinus and nasal cavity cancer; parathyroid cancer; penile cancer; pharyngeal cancer; pheochromocytoma; childhood pheochromocytoma; pituitary tumor; plasma cell neoplasm/multiple myeloma; pleuropulmonary blastoma; pregnancy and breast cancer; primary peritoneal cancer; prostate cancer; rectal cancer; recurrent cancer; renal cell (kidney) cancer; retinoblastoma; rhabdomyosarcoma, childhood; salivary gland cancer; sarcoma (e.g., childhood rhabdomyosarcoma, childhood vascular tumors, Ewing sarcoma, Kaposi sarcoma, osteosarcoma (bone cancer), soft tissue sarcoma, uterine sarcoma); Sézary syndrome; skin cancer; childhood skin cancer; small cell lung cancer; small intestine cancer; soft tissue sarcoma; squamous cell carcinoma of the skin; squamous neck cancer with occult primary, metastatic; T-cell lymphoma, cutaneous (e.g., mycosis fungoides and Sèzary syndrome); testicular cancer; childhood testicular cancer; throat cancer (e.g., nasopharyngeal cancer, oropharyngeal cancer, hypopharyngeal cancer); thymoma and thymic carcinoma; thyroid cancer; transitional cell cancer of the renal pelvis and ureter; ureter and renal pelvis, transitional cell cancer; urethral cancer; uterine cancer, endometrial; uterine sarcoma; vaginal cancer; childhood vaginal cancer; vascular tumors; vulvar cancer; and Wilms tumor and other childhood kidney tumors.

In some embodiments, a method disclosed herein comprises:
a) conditioning a NRGS mouse on Day 0,
b) engrafting a population of about 5×10⁶-1×10⁷ peripheral blood mononuclear cells (PBMCs) to the NRGS mouse on Day 1, wherein the population of PBMCs is from a stem cell-mobilized healthy human, is T-cell depleted, and does not comprise cancer cells, and
c) engrafting a population of lymphocytes from the human to the NRGS mouse on Day 28, wherein the population of lymphocytes comprises about 1×10⁶ CD4⁺ T cells, optionally, the population of lymphocytes further comprises about 5×10⁵ CD19⁺ B cells.

In some embodiments, a method disclosed herein comprises the steps of:
a) conditioning a NRGS mouse on Day 0;
b) incubating a population of about 5×10⁶-1×10⁷ peripheral blood mononuclear cells (PBMCs) with a compound, a pharmaceutically acceptable salt thereof, or a composition of International Patent Application No. PCT/US2022/071082 (International Publication No. WO 2022/192899), PCT/US2022/74903 (International Publication No. WO 2023/019242), and/or PCT/US2022/74908 (International Publication No. WO 2023/019244) for 5 days, wherein the population of PBMCs is from a stem cell-mobilized healthy human, is not T-cell depleted, and does not comprise cancer cells; and
c) engrafting the PBMCs to the conditioned NRGS mouse on Day 5.

In some embodiments, a method disclosed herein comprises the steps of:
a) conditioning a NRGS mouse on Day 0;
b) engrafting a population of about 5×10⁶-1×10⁷ peripheral blood mononuclear cells (PBMCs) to the NRGS mouse on Day 1, wherein the population of PBMCs is from a stem cell-mobilized healthy human, is T-cell depleted, and does not comprise cancer cells;
c) incubating a population of human lymphocytes from the heathy human with a compound, a pharmaceutically acceptable salt thereof, or a composition of International Patent Application No. PCT/US2022/071082 (International Publication No. WO 2022/192899), PCT/US2022/74903 (International Publication No. WO 2023/019242), and/or PCT/US2022/74908 (International Publication No. WO 2023/019244) for 5 days, wherein the population of human lymphocytes comprises about 1×10⁶ CD4⁺ T cells, optionally, the population of human lymphocytes further comprises about 5×10⁵ CD19⁺ B cells; and
d) engrafting the population of human lymphocytes to the conditioned NRGS mouse on Day 7.

### Non-Human Animals and Non-Human Animal Models

Also provided herein, among other things, are non-human animals comprising a functional human immune system.

A functional human immune system comprises mature human T cells and mature human B cells. Whether a non-human animal has a functional human immune system can be evaluated, for example, by detecting and/or quantifying human T cells (e.g., human CD3⁺ T cells) in the non-human animal, for example, using FACS; detecting and/or quantifying human B cells (e.g., mature human B cells) in the non-human animal, for example, using FACS; detecting and/or quantifying human NK cells (e.g., mature human NK cells) in the non-human animal, for example, using FACS; detecting antibody response rate to a human antigen in one or more non-human animals in accordance with the present disclosure, e.g., as described in Example 2; and/or by detecting human antibody production by the non-human animal. In some embodiments, a functional human immune system comprises mature human T cells and mature human B cells in a ratio approximately equivalent (such as within approximately 30%, such as within approximately 29%, 28%, 26%, 25%, 24%, 23%, 22%, 21%, 20%, 19%, 18%, 17%, 16%, 15%, 14%, 13%, 12%1, 1%, 10%, 9%, 8%, 7%, 6%, or 5%) to that found in humans (e.g., healthy humans), such as the one or more humans who donated the population of cells in an engraftment described herein. For example, in humans, a mature T cell to mature B cell ratio may increase with age, e.g., from a ratio of about 2 (e.g., about 2 mature T cells:1 mature B cell) to about 4-5 or greater in adults. In some embodiments, the non-human animal comprises mature human T cells and mature human B cells in a ratio of about 1.5, about 1.75, about 2, about 2.25, about 2.5, about 2.75, about 0.3, about 3.25, about 3.5, about 3.75, about 4, about 4.25, about 4.5, about 4.75, about 5, about 5.25, about 5.5, about 5.75, or about 6. In some embodiments, the non-human animal comprises mature human T cells and mature human B cells in a ratio of between about 1.5 and 6, between about 1.5 and 5, between about 1.5 and 4.5, between about 1.5 and 4, between about 1.5 and 3.5, between about 1.5 and 3, between about 1.5 and 2.5, between about 2 and 6, between about 2 and 5, between about 2 and 4, or between about 2 and 3. In some embodiments, a functional human immune system comprises human CD4⁺ T cells and human CD8⁺ T cells in a ratio approximately equivalent to that found in humans (e.g., healthy humans), such as the one or more humans who donated the population of cells in an engraftment described herein. In some embodiments, the non-human animal comprises human CD4⁺ T cells and human CD8⁺ T cells in a ratio of at least 1. In some embodiments, a ratio of human CD4⁺ T cells to human CD8⁺ T cells in the non-human animal is about 1, about 1.25, about 1.5, about 1.75, about 2, about 2.25, about 2.5, about 2.75, about 3, about 3.25, about 3.5, about 3.75, about 4, or greater than about 4. In some embodiments a ratio of human CD4⁺ T cells to human CD8⁺ T cells in the non-human animal is between about 1 and 4, such as between about 1 and 3.75, between about 1 and 3.5, between about 1 and 3.25, between about 1 and 3, between about 1 and 2.75, between about 1 and 2.5, between about 1 and 2.25, between about 1 and 2, between about 1 and 1.75, between about 1 and 1.5, or between about 1 and 1.25.

In one aspect, a non-human animal disclosed herein:
a) lacks endogenous mature T cells; and
b) comprises a functional human immune system generated from an engraftment of a population of human mononuclear cells, human polymorphonuclear leukocytes, or both (such as from one or more humans, such as 1, 2, 3, 4, or 5 or more humans), wherein the engraftment lacks cancer cells (e.g., human cancer cells).

Also provided herein, among other things, are immune cell-engrafted non-human animals. In some embodiments, the non-human animals comprise a functional human immune system.

In one aspect, a non-human animal disclosed herein:
a) lacks endogenous mature T cells; and
b) comprises an engraftment of a population of human mononuclear cells, human polymorphonuclear leukocytes, or both (such as from one or more healthy humans, such as 1, 2, 3, 4, or 5 or more healthy humans), wherein the engraftment lacks cancer cells (e.g., human cancer cells).

In another aspect, a non-human animal is generated by any one or more methods disclosed herein.

Also provided herein, among other things, are non-human animal models. In one aspect, a non-human animal model comprises any two or more (such as any 2, 3, 4, 5, 6, 7, 8, 9, 10, 11, 12, 13, 14, 15, 16, 17, 18, 19, 20, 2-50, 2-40, 2-30, 2-20, 2-10, or 2-5) non-human animals (e.g., a mouse, such as a NRGS mouse or a NSGS mouse) described herein. In some embodiments, the engraftment in each non-human animal is from the same human. In some embodiments, the engraftment in each non-human animal is from a different human.

In another aspect, a non-human animal model is generated by any one or more methods disclosed herein.

The non-human animal can be any one or more of non-human animals described herein, e.g., in Methods of Generating Non-Human Animals and Non-Human Animal Models.

In some embodiments, the non-human animal disclosed herein (e.g., a mouse, such as a NRGS mouse or a NSGS mouse) is conditioned with radiation, chemotherapy, or a combination thereof, according to any of the embodiments described herein, e.g., in Methods of Generating Non-Human Animals and Non-Human Animal Models, in particular, Conditioning.

The human can be any one or more of humans described herein, e.g., in Methods of Generating Non-Human Animals and Non-Human Animal Models.

The population of human mononuclear cells, polymorphonuclear leukocytes, or both can be any one or more (such as any 1, 2, 3, 4, 5, 6, 7, 8, 9, or 10 or more) populations of human mononuclear cells, polymorphonuclear leukocytes, or both described herein, e.g., in Methods of Generating Non-Human Animals and Non-Human Animal Models.

In some embodiments, the non-human animal disclosed herein further comprises an engraftment of a population of human lymphocytes. The population of human lymphocytes can be any one or more (such as any 1, 2, 3, 4, 5, 6, 7, 8, 9, or 10 or more) of populations of human lymphocytes described herein, e.g., in Methods of Generating Non-Human Animals and Non-Human Animal Models.

In some embodiments, a non-human animal (e.g., a NRGS or NSGS mouse) is administered any one or more TIM receptor agonists described herein, e.g., in Methods of Generating Non-Human Animals and Non-Human Animal Models, in particular, TIM Receptor Agonists. In some embodiments, a population of human mononuclear cells, human polymorphonuclear leukocytes, or both is incubated with any one or more TIM receptor agonists described herein, e.g., in Methods of Generating Non-Human Animals and Non-Human Animal Models, in particular, TIM Receptor Agonists. In some embodiments, a population of human lymphocytes (e.g., CD4⁺ T cells and/or CD19⁺ B cells) is incubated with any one or more TIM receptor agonists described herein, e.g., in Methods of Generating Non-Human Animals and Non-Human Animal Models, in particular, TIM Receptor Agonists.

In some embodiments, a population of human mononuclear cells, human polymorphonuclear leukocytes, or both does not comprise cancer cells. In some embodiments, a population of human mononuclear cells, human polymorphonuclear leukocytes, or both and a population of human lymphocytes do not comprise cancer cells. In some embodiments, the non-human animal lacks human cancer cells. In some embodiments, cancer cells are any of the cancer cells described herein, e.g., in Methods of Generating Non-Human Animals and Non-Human Animal Models, in particular, Lacking Cancer Cells.

In some embodiments of any of the methods or non-human animals described herein, at least 90% of CD3 cells in a non-human animal, e.g., comprising a functional human immune system, are human CD3 cells, for example, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% of CD3 cells in the non-human animal, e.g., comprising a functional human immune system, are human CD3 cells.

In some embodiments of any of the methods or non-human animals described herein, CD45⁺ cells in a non-human animal described herein lack non-human (e.g., mouse) CD4⁺ T cells, lack non-human (e.g., mouse) CD19⁺ B cells, or both. In some embodiments, the CD45⁺ cells lack non-human (e.g., mouse) CD4⁺ T cells. In some embodiments, the CD45⁺ cells lack non-human (e.g., mouse) CD19⁺ B cells. In some embodiments, the CD45⁺ cells lack non-human (e.g., mouse) CD4⁺ T cells and lack non-human (e.g., mouse) CD19⁺ B cells.

In some embodiments of any of the methods or non-human animals described herein, CD4⁺ cells in a non-human animal described herein comprise only human CD4⁺ T cells, and/or CD19⁺ B cells in a non-human animal described herein comprise only human CD19⁺ B cells.

In some embodiments of any of the methods or non-human animals described herein, at least 30% of CD45 cells in a non-human animal, e.g., comprising a functional human immune system, are human CD45 cells, for example, at least 32%, at least 35%, at least 38%, at least 40%, at least 42%, at least 45%, at least 48%, or at least 50% of CD45 cells in the non-human animal, e.g., comprising a functional human immune system, are human CD45 cells.

In some embodiments of any of the methods or non-human animals described herein, at least about 5% of CD45⁺ cells in a non-human animal, e.g., comprising a functional human immune system, described herein are human CD45⁺ cells, for example, at least about: 10%, 15%, 20%, 25%, or 30% of CD45⁺ cells in the non-human animal are human CD45⁺ cells.

In some embodiments of any of the methods or non-human animals described herein, at most about 90% of CD45⁺ cells in a non-human animal, e.g., comprising a functional human immune system, described herein are human CD45⁺ cells, for example, at most about: 85%, 80%, 75%, 70%, or 65% of CD45⁺ cells in the non-human animal are human CD45⁺ cells.

In some embodiments of any of the methods or non-human animals described herein, about 5-90% of CD45⁺ cells in a non-human animal, e.g., comprising a functional human immune system, described herein are human CD45⁺ cells, for example, about: 5-85%, 10-85%, 10-80%, 15-80%, 15-75%, 20-75%, 20-70%, 25-70%, or 25-65% of CD45⁺ cells in the non-human animal are human CD45⁺ cells.

In some embodiments of any of the methods or non-human animals described herein, at least about 5% of CD45⁺CD4⁺ cells in a non-human animal, e.g., comprising a functional human immune system, described herein are human CD4⁺ cells, for example, at least about: 10%, 15%, 20%, 25%, or 30% of CD45⁺CD4⁺ cells in the non-human animal are human CD4⁺ cells. In some embodiments, at least about 20% of CD45⁺CD4⁺ cells in the non-human animal are human CD4⁺ cells. In some embodiments, at least about 25% of CD45⁺CD4⁺ cells in the non-human animal are human CD4⁺ cells.

In some embodiments of any of the methods or non-human animals described herein, at most about 60% of CD45⁺CD4⁺ cells in a non-human animal, e.g., comprising a functional human immune system, described herein are human CD4⁺ cells, for example, at most about: 55%, 50%, 45%, 40%, or 35% of CD45⁺CD4⁺ cells in the non-human animal are human CD4⁺ cells. In some embodiments, at most about 40% of CD45⁺CD4⁺ cells in the non-human animal are human CD4⁺ cells. In some embodiments, at most about 35% of CD45⁺CD4⁺ cells in the non-human animal are human CD4⁺ cells.

In some embodiments of any of the methods or non-human animals described herein, about 5-60% of the CD45⁺CD4⁺ cells in a non-human animal, e.g., comprising a functional human immune system, described herein are human CD4⁺ cells, for example, about: 5-55%, 10-55%, 10-50%, 10-45%, 15-45%, 15-40%, 20-40%, 20-35%, 25-35%, or 25-30% of the CD45⁺CD4⁺ cells in the non-human animal are human CD4⁺ cells. In some embodiments, about 25-35% of the CD45⁺CD4⁺ cells in the non-human animal are human CD4⁺ cells. In some embodiments, about 25-30% of the CD45⁺CD4⁺ cells in the non-human animal are human CD4⁺ cells.

In some embodiments of any of the methods or non-human animals described herein, at least about 1% of the CD45⁺CD19⁺ cells in a non-human animal, e.g., comprising a functional human immune system, described herein are human CD19⁺ cells, for example, at least about: 2%, 3%, 4%, 5%, 6%, 7%, 8%, or 9% of the CD45⁺CD19⁺ cells in the non-human animal are human CD19⁺ cells. In some embodiments, at least about 8% of the CD45⁺CD19⁺ cells in the non-human animal are human CD19⁺ cells. In some embodiments, at least about 9% of the CD45⁺CD19⁺ cells in the non-human animal are human CD19⁺ cells.

In some embodiments of any of the methods or non-human animals described herein, at most about 40% of the CD45⁺CD19⁺ cells in a non-human animal, e.g., comprising a functional human immune system, described herein are human CD19⁺ cells, for example, at most about: 35%, 30%, 25%, 20%, 18%, or 16% of the CD45⁺CD19⁺ cells in the non-human animal are human CD19⁺ cells. In some embodiments, at most about 20% of the CD45⁺CD19⁺ cells in the non-human animal are human CD19⁺ cells. In some embodiments, at most about 16% of the CD45⁺CD19⁺ cells in the non-human animal are human CD19⁺ cells.

In some embodiments of any of the methods or non-human animals described herein, about 1-40% of the CD45⁺CD19⁺ cells in a non-human animal, e.g., comprising a functional human immune system, described herein are human CD19⁺ cells, for example, about: 1-35%, 2-35%, 2-30%, 4-30%, 4-25%, 6-25%, 6-20%, 8-20%, 8-16%, 9-16%, or 9-12% of the CD45⁺CD19⁺ cells in the non-human animal are human CD19⁺ cells. In some embodiments, about 9-16% of the CD45⁺CD19⁺ cells in the non-human animal are human CD19⁺ cells. In some embodiments, about 9-12% of the CD45⁺CD19⁺ cells in the non-human animal are human CD19⁺ cells.

In some embodiments of any of the methods or non-human animals described herein, a non-human animal, e.g., comprising a functional human immune system, described herein has a CD8⁺/CD4⁺ ratio approximately equivalent to that found in humans (e.g., healthy humans), such as the one or more humans who donated the population of cells in an engraftment described herein. In some embodiments of any of the methods or animals described herein, a non-human animal, e.g., comprising a functional human immune system, described herein has a CD8⁺/CD4⁺ ratio of about 1/4-1/2, for example, about: 1/4, 1/3, 5/12, or 1/2.

In some embodiments of any of the methods or non-human animals described herein, the non-human animal further comprises human cancer cells, e.g., from a human patient-derived xenograft (PDX). In some embodiments, human cancer cells described herein are allogenic to a functional human immune system described herein. In some embodiments, human cancer cells described herein are syngeneic (e.g., autologous) to a functional human immune system described herein.

In some embodiments, cancer cells are hematological cancer cells. Examples of hematological malignancies include leukemia, such as acute lymphocytic leukemia (ALL), chronic lymphocytic leukemia (CLL), acute myeloid leukemia (AML) or chronic myeloid leukemia (CML), myeloma, and lymphoma. In other embodiments, cancer cells are solid tumor cancer cells. Examples of solid tumors include tumors of the breast, lung, prostate, colon, bladder, ovary, kidney, stomach, colon, rectum, testes, head and/or neck, pancreas, brain and skin. Other non-limiting examples of cancers are described in Lacking Cancer Cells.

### Uses of the Animals and Animal Models Disclosed Herein

Also provided herein, among other things, are methods of determining immunogenicity of an antigen or an immunogenic fragment thereof, or an antigenic therapy, comprising:
administering the antigen or the immunogenic fragment thereof, or the antigenic therapy to any one or more non-human animals described herein or one or more non-human animals in a non-human animal model disclosed herein, and
detecting an immune response to the antigen or the immunogenic fragment thereof, or the antigenic therapy in the one or more non-human animals.

Also provided herein, among other things, are methods of identifying an agent for modulating (e.g., reducing) an immune response (e.g., an immune response to an antigen or an immunogenic fragment thereof, or an antigenic therapy), comprising:
administering an antigen or an immunogenic fragment thereof, or an antigenic therapy to any one or more non-human animals described herein or one or more non-human animals in a non-human animal model disclosed herein,
administering an agent suspected to modulate (e.g., reduce) an immune response of the one or more non-human animals, and
detecting an immune response to the antigen or the immunogenic fragment thereof, or the antigenic therapy in the one or more non-human animals.

In some embodiments, a method described herein comprises predicting a clinical immunogenicity rate of an antigen or an immunogenic fragment thereof, or an antigenic therapy, comprising:
administering the antigen or the immunogenic fragment thereof, or the antigenic therapy thereof to one or more non-human animals described herein or one or more non-human animals in a non-human animal model described herein, and
detecting an immune response to the antigen or the immunogenic fragment thereof, or the antigenic therapy in the one or more non-human animals.

In some embodiments, an immune response described herein comprises an innate response (e.g., activated by pathogen-associated molecular patterns (PAMPs) and/or damage-associated molecular patterns (DAMPs)), an adaptive response, or both. In some embodiments, the immune response comprises an innate response. In some embodiments, the immune response comprises an adaptive response. In some embodiments, the immune response comprises an innate response and an adaptive response.

In some embodiments, an immune response described herein comprises a specific immune response, a non-specific immune response, or both. In some embodiments, the immune response comprises a specific immune response. In some embodiments, the immune response comprises a non-specific immune response. In some embodiments, the immune response comprises a specific immune response and a non-specific immune response.

In some embodiments, a method described herein comprises attributing an immunogenicity to a property of an antigen. In some embodiments, a property of an antigen comprises formulation, aggregation, or both.

In some embodiments, the one or more non-human animals comprise two or more non-human animals, e.g., ≥3, ≥4, ≥5, ≥6, ≥7, ≥8, ≥9, ≥10, ≥12, ≥15, ≥20, ≥25, ≥30, ≥40, ≥50, ≥60, ≥70, ≥80, ≥90, or ≥100 non-human animals.

In some embodiments, engraftment in each non-human animal is from the same human. In some embodiments, engraftment in each non-human animal is from a different human.

As used herein, an “immunogenic fragment” of an antigen refers to a fragment of the antigen that induces an immune response to the antigen. An immunogenic fragment of an antigen may induce an immune response in a subject that is similar in extent to the immune response induced by the antigen itself but need not induce the same extent of immune response as the antigen itself.

In some embodiments, an antigen comprises a therapeutic. In some embodiments, a therapeutic comprises a polynucleotide, a polypeptide, a cell, a tissue, or any combination of the foregoing. In some embodiments, detecting an immune response to an antigen or an immunogenic fragment thereof, or an antigenic therapy comprises detecting antibodies to the antigen. In some embodiments, detecting an immune response to an antigen comprises quantifying antibodies to the antigen or the immunogenic fragment thereof, or the antigenic therapy.

In some embodiments, an antigenic therapy is an antibody therapy (e.g., monoclonal antibody therapy), including chimeric, humanized and fully-human antibody therapies. Non-limiting examples of antibody therapies include anti-tumor necrosis factor (anti-TNF) therapies, such as adalimumab (Humira®; for rheumatoid arthritis, juvenile idiopathic arthritis, psoriatic arthritis, ankylosing spondylitis, Crohn's disease, ulcerative colitis, plaque psoriasis, hidradenitis suppurativa, uveitis) and infliximab (Remicade®, for Crohn's disease, pediatric Crohn's disease, ulcerative colitis, pediatric ulcerative colitis, rheumatoid arthritis, ankylosing spondylitis, psoriatic arthritis, plaque psoriasis), golimumab (Simponi®, for rheumatoid arthritis, psoriatic arthritis, ankylosing spondylitis, polyarticular juvenile idiopathic arthritis), etanercept (Enbrel®, for rheumatoid arthritis, polyarticular juvenile idiopathic arthritis, psoriatic arthritis, ankylosing spondylitis, plaque psoriasis) and certolizumab pegol (Cimzia®, for Crohn's disease, rheumatoid arthritis, psoriatic arthritis, ankylosing spondylitis, non-radiographic axial spondyloarthritis, plaque psoriasis).

In some embodiments, an antigenic therapy is a protein replacement therapy, for example, enzyme replacement therapy. Examples of protein replacement therapies include replacement therapies for coagulation disorders, such as Factor VIII and Factor IX for hemophilia A and B; enzyme replacement therapies for lysosomal storage diseases, such as alglucosidase alfa (Myozyme® and Lumizyme®) for Pompe disease; alpha-L-iduronidase for Hurler syndrome; and adenosine deaminase for adult-type adenosine deaminase deficiency.

In some embodiments, an antigenic therapy is a gene therapy. Gene therapies typically work by one of the following three mechanisms: (1) by supplying a subject with a healthy copy of a disease-causing gene (as does voretigene neparvovec-rzyl (Luxturna®), for example); (2) by inactivating a disease-causing gene (as may ASOs and siRNA, for example); or (3) by introducing a gene into the body to help treat a disease. Gene therapies include DNA (e.g., antisense oligonucleotides (ASOs)) and/or RNA (e.g., siRNA), which can be delivered to a subject in vivo or ex vivo via a variety of products. In vivo gene delivery products include plasmid DNA, viral vectors (e.g., AAV, such as AAV9) and non-viral vectors, such as bacterial vectors or lipid nanoparticles. Other examples of non-viral vectors suitable for in vivo gene delivery include exosomes, polymeric particles, inorganic particles and lipid-polymer hybrid particles. Ex vivo gene delivery products include subject-derived cellular gene therapy products. Gene therapies also include gene editing technologies, such as CRISPR. Gene editing technologies, such as CRISPR, can conveniently be delivered to a subject via any of the products for in vivo gene delivery described herein. Non-limiting examples of gene therapies include voretigene neparvovec-rzyl (Luxturna®, for retinal dystrophy); and onasemnogene abeparvovec-xioi (Zolgensma®, for pediatric spinal muscular atrophy).

In some embodiments, a gene therapy comprises DNA and/or RNA and a viral vector. In some embodiments, a viral vector is derived from an adeno-associated virus (AAV), such as a recombinant AAV. In some embodiments, an AAV is AAV9. Other examples of viral vectors suitable for use in the context of the present disclosure include viral vectors derived from retrovirus, herpes virus, adenovirus, lentivirus, rabies virus, lentivirus, VSV, poxvirus (e.g., vaccinia virus, variola virus, canarypox), reovirus, semliki forest virus, yellow fever virus, sindbis virus, togavirus, baculovirus, bacteriophages, alphavirus, and flavavirus.

In some embodiments, an antigenic therapy is a cellular therapy. An example of a cellular therapy is axicabtagene ciloleucel (Yescarta®, for relapsed or refractory large B-cell lymphoma). Another example of a cellular therapy is CAR-T cells.

Alloantigens, antigens present in some but not all individuals of a species and recognized as foreign by those that lack it, are often the basis for graft rejection reactions. Examples of alloantigens include, but are not limited to, major histocompatibility complex (MHC) class I and class II antigens, minor histocompatibility antigens, endothelial glycoproteins, such as blood group antigens, and carbohydrate determinants.

In some embodiments, immunogenicity is determined using a measurable immune response, e.g., to an antigen, such as measurable antibody production in response to an antigen. ELISA and/or other activity assays known in the art can be used to measure antibody production indicative of immunogenicity.

In some embodiments, immunogenicity is determined in a general manner, for example by assessing:
immunological hyperactivity;
anti-inflammatory cytokine release;
neutralization of immune cells (e.g., macrophages, neutrophils, eosinophils, T cells and B cells);
the number of regulatory T cells and/or activity of tolerogenic T cells; or
an increase in the number of regulatory B cells, or
any combinations of the foregoing.

Non-limiting examples of tolerogenic T cells include FoxP3⁺/CD4⁺ T cells, CD4⁺/CD25^{hi}/Foxp3⁺/CTLA4⁺/Tim3⁺ T cells, CD4⁺/CD25^{hi}/Foxp3⁺/CTLA4⁺/NRP1⁺/ICOS⁻ T cells, and CD4⁺/CD25^{hi}/Foxp3⁺/CTLA4⁺/Tim3⁺/NRP1⁺/ICOS⁻ T cells.

Non-limiting examples of regulatory B cells include CD19⁺/CD71⁺/IgM⁺ B cells and CD19⁺/CD71⁺/IgM⁺/CD24⁺/CD38⁺/CD27⁺ B cells.

In some embodiments, immunogenicity is determined in an antigen-specific manner, for example:
an increase in the number of antigen-specific regulatory T cells;
a decrease in antigen-specific antibody titer and/or number of B cells, including
antigen-specific memory B cells;
a decrease in IL-6 and/or IL-17;
an increase in TGF-beta, IL-10, IL-35, CD40, CD80 and/or CD86;
hyporesponsiveness following re-challenge with an antigen; or
an increase in the number of antigen-specific regulatory B cells (e.g., CD19⁺/CD71⁺/IgM⁺/CD24⁺/CD38⁺/CD27⁺ B cells; and/or CD19⁺/CD71⁺/IgM⁺ B cells), or
any combinations of the foregoing.

Non-limiting examples of antigen-specific regulatory T cells include CD4⁺/CD25^{hi}/Foxp3⁺/CTLA4⁺/Tim3⁺ T cells, CD4⁺/CD25^{hi}/Foxp3⁺/CTLA4⁺/NRP1⁺/ICOS⁻ T cells, CD4⁺/CD25^{hi}/Foxp3⁺/CTLA4⁺/Tim3⁺/NRP1⁺/ICOS⁻ T cells, and CD4⁺/FoxP3⁺ T cells.

Techniques for evaluating these indicators (such as the indicators of an immune response or of immunogenicity as described herein) are known in the art and described in, for example, International Patent Application Nos. PCT/US2022/071082 (International Publication No. WO 2022/192899), PCT/US2022/74903 (International Publication No. WO 2023/019242), and/or PCT/US2022/74908 (International Publication No. WO 2023/019244), the contents of which are incorporated herein by reference.

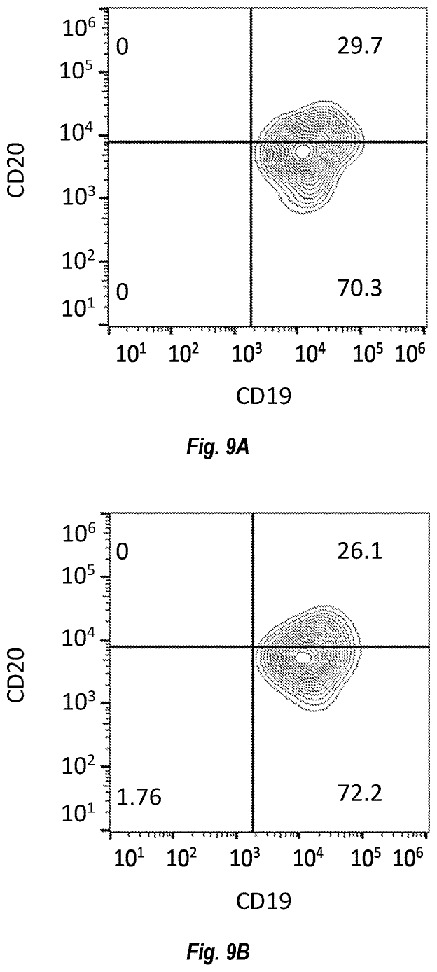

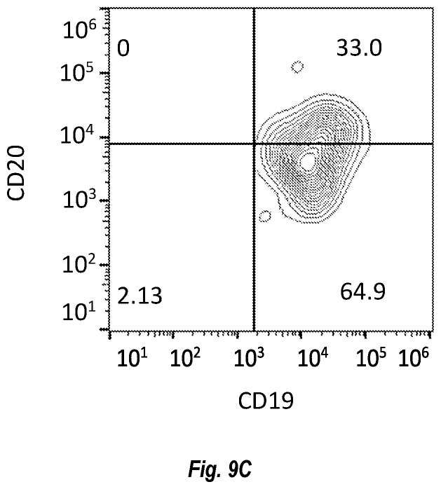

In some embodiments of the disclosed methods (e.g., methods of determining immunogenicity of an antigen or an immunogenic fragment thereof, or an antigenic therapy; methods of identifying an agent for modulating immune intolerance; and/or methods of identifying an agent for modulating (e.g., reducing) an immune response (e.g., an immune response to an antigen or an immunogenic fragment thereof, or an antigenic therapy)), the non-human animal comprises human CD20+ B cells. CD20 can be expressed on mature (e.g., activated, antibody-producing) B cells. In some embodiments, expansion of human CD20+ B cell populations can occur following antigen exposure in non-human animals disclosed herein. In particular embodiments, the human CD20+ B cells are present in the non-human animal at a greater level after a step of administering an antigen or an immunogenic fragment thereof, or an antigenic therapy to the one or more non-human animals, as compared to before the administering step, consistent with B cell maturation following antigen exposure (FIGS. 9A-9B, Example 3).

Because the methods described herein for generating non-human animals with functional human immune systems are not associated with xenorejection and/or graft-versus-host disease, the non-human animals and animal models described herein can be used to evaluate graft-versus-tumor activity, e.g., in a non-human animal, such as a mouse, that has human CD4⁺ and human CD8⁺ T cells, and to study human-derived tumors in the non-human animals.

Also provided herein, among other things, are methods of evaluating cancer-immune cell interaction, comprising:
engrafting xenograft (e.g., a human patient-derived xenograft (PDX)) to any one or more non-human animals disclosed herein, and
evaluating the cancer-immune cell interaction.

Also provided herein, among other things, are methods of evaluating a cancer-immune cell interaction, comprising:
engrafting cancer cells (e.g., from a human patient-derived cancer cell line) to any one or more non-human animals disclosed herein, and
evaluating the cancer-immune cell interaction.

In some embodiments, evaluating the cancer-immune cell interaction comprises measuring tumor size and/or evaluating immune-population profile in blood and/or tumor (e.g., measuring percentage of hCD3⁺/hCD45⁺, hCD4⁺/hCD3⁺, CD8⁺/hCD3⁺, natural killer T (NKT)/hCD3⁺, Treg/hCD4⁺, dysfunctional hCD3⁺, antigen-presenting cells (e.g., hCD11b⁺, hCD14⁺, MSDC⁺, etc.), or a combination thereof).

Also provided herein, among other things, are methods of evaluating an effect of an anti-cancer agent, comprising:
administering the anti-cancer agent to any one or more non-human animals disclosed herein, wherein the one or more non-human animals have cancer (e.g., tumor); and
evaluating the effect of the anti-cancer agent.

Also provided herein, among other things, are methods of evaluating an effect of an anti-cancer agent, comprising:
engrafting xenograft (e.g., a human patient-derived xenograft (PDX), organoid tumor) to any one or more non-human animals disclosed herein,
administering the anti-cancer agent to the one or more non-human animals; and
evaluating the effect of the anti-cancer agent.

Also provided herein, among other things, are methods of evaluating an effect of an anti-cancer agent, comprising:
engrafting cancer cells (e.g., from a human patient-derived cancer cell line) to any one or more non-human animals disclosed herein,
administering the anti-cancer agent to the one or more non-human animals; and
evaluating the effect of the anti-cancer agent.

In some embodiments, a xenograft described herein is a cancer xenograft, e.g., a human PDX. In some embodiments, the human patient is treatment-naïve. In some embodiments, the human patient is treatment-resistant. In some embodiments, the human PDX and a functional human immune system described herein are allogenic. In some embodiments, the human PDX and a functional human immune system described herein are syngeneic (e.g., autologous).

In some embodiments, a human PDX described herein is from a primary patient sample. In some embodiments, the human PDX is from an archived tumor sample. In some embodiments, the human PDX has been passaged as a xenograft for at least 1 generation, e.g., for at least 2, 3, 4 or 5 generations. In some embodiments, the human PDX has been passaged as a xenograft for no more than 5 generations, e.g., for no more than 4, 3, 2 or 2 or 1 generation. In some embodiments, the human PDX has been passaged in culture for at least 1 generation, e.g., for at least 2, 3, 4 or 5 generations. In some embodiments, the human PDX has been passaged in culture for no more than 5 generations, e.g., for no more than 4, 3, 2 or 2 or 1 generation.

In some embodiments, a method described herein determines efficacy and/or safety of at least one anti-cancer agent (e.g., at one or more dosing regimens) for treating the tumor represented by the xenograft (e.g., human PDX).

In some embodiments, the method determines efficacy and/or safety of at least two anti-cancer agents (e.g., at one or more dosing regimens) for treating the tumor. In some embodiments, the method further comprises comparing the efficacy and/or safety of the at least two anti-cancer agents for treating the tumor represented by the xenograft (e.g., human PDX).

In some embodiments, the at least two anti-cancer agents are administered either as single agent or as a combination. In some embodiments, the efficacy and/or safety of the combination is compared to the efficacy and/or safety of the single agents (e.g., to determine if the combination shows one or more synergistic effects).

In some embodiments, the method determines efficacy and/or safety of a plurality of anti-cancer agents (e.g., at one or more dosing regimens) for treating the tumor represented by the xenograft (e.g., human PDX). In some embodiments, the method further comprises comparing the efficacy and/or safety of each one of said plurality of anti-cancer agents. In some embodiments, the method further comprises ranking the efficacy and/or safety of each one of said plurality of anti-cancer agents.

In some embodiments, an anti-cancer agent described herein comprises an anti-cancer compound (e.g., a preclinical chemotherapeutic reagent). In some embodiments, an anti-cancer agent described herein comprises a polynucleotide, a polypeptide, a cell, a tissue, or any combination of the foregoing.

In certain embodiments, an anti-cancer agent described herein is an immunomodulator, such as a modulator (e.g., inhibitor, agonist) of an immune checkpoint. Examples of immune checkpoints include programmed cell death protein 1 (PD-1), programmed cell death ligand 1 (PD-L1), cytotoxic T-lymphocyte-associated protein 4 (CTLA-4), lymphocyte-activation gene 3 (LAG-3), T cell immunoglobulin and mucin domain molecule (TIM, e.g., TIM-3 and/or TIM-4), TIGIT, VISTA and KLRG-1. In some embodiments, an anti-cancer agent is an immune checkpoint inhibitor, such as a PD-1, PD-L1 or CTLA4 inhibitor. In some embodiments, an anti-cancer agent is a TIM receptor agonist, including any of those described herein.

In some embodiments, an anti-cancer agent described herein is an antibody or antigen-binding fragment thereof (e.g., a monoclonal antibody), including chimeric, humanized and fully-human antibody therapies. In some embodiments, an anti-cancer antibody (e.g., monoclonal antibody) described herein engages components of the functional human immune system (e.g., exerting complement dependent cytotoxicity (CDC), antibody-dependent cellular phagocytosis (ADCP), antibody-dependent cellular cytotoxicity (ADCC), or a combination thereof).

In some embodiments, an anti-cancer agent described herein is a vaccine. In some embodiments, the vaccine comprises a cancer-specific antigen (e.g., a tumor-specific antigen). In some embodiments, the vaccine increases a cancer-specific (e.g., tumor-specific) T cell response in a non-human disclosed herein.

In some embodiments, an anti-cancer agent described herein (e.g., an antibody or antigen-binding fragment) is incorporated into a cell-based therapy. In some embodiments, the anti-cancer agent is an engineered T cell receptor. In some embodiments, the anti-cancer agent is a chimeric antigen receptor (CAR) (e.g., expressed on a T (CAR-T) cell, natural killer (CAR-NK) cell, or macrophage (CAR-M) cell).

In some embodiments, an anti-cancer therapy is a gene therapy, for example by inactivating a cancer-causing gene (as may ASOs and siRNA, for example), or by introducing a gene into the body to help treat a cancer. Anti-cancer gene therapies include DNA (e.g., antisense oligonucleotides (ASOs)) and/or RNA (e.g., siRNA), which can be delivered to a subject in vivo or ex vivo via a variety of products. In vivo gene delivery products include plasmid DNA, viral vectors (e.g., AV or AAV, such as AAV9) and non-viral vectors, such as bacterial vectors or lipid nanoparticles. Other examples of non-viral vectors suitable for in vivo gene delivery include exosomes, polymeric particles, inorganic particles and lipid-polymer hybrid particles. Ex vivo gene delivery products include subject-derived cellular gene therapy products. Gene therapies also include gene editing technologies, such as CRISPR. Gene editing technologies, such as CRISPR, can conveniently be delivered to a subject via any of the products for in vivo gene delivery described herein.

In some embodiments, an anti-cancer gene therapy comprises DNA and/or RNA and a viral vector. In some embodiments, a viral vector is derived from an adeno-associated virus (AAV), such as a recombinant AAV. In some embodiments, an AAV is AAV9. Other examples of viral vectors suitable for use in the context of the present disclosure include viral vectors derived from retrovirus, herpes virus, adenovirus, lentivirus, rabies virus, lentivirus, VSV, poxvirus (e.g., vaccinia virus, variola virus, canarypox), reovirus, semliki forest virus, yellow fever virus, sindbis virus, togavirus, baculovirus, bacteriophages, alphavirus, and flavivirus.

Also provided herein, among other things, are methods of evaluating an effect of an immunomodulatory agent, comprising:
administering the immunomodulatory agent to any one or more non-human animals disclosed herein; and evaluating the effect of the immunomodulatory agent.

Examples of immunomodulatory agents include checkpoint inhibitors (e.g., PD-1 inhibitors, such as pembrolizumab, nivolumab, cemiplimab; PD-L1 inhibitors, such as atezolizumab, avelumab, durvalumab; CTLA-4 inhibitors; such as ipilimumab, tremelimumab; PD-L2 inhibitors; TIM inhibitors; LAG-3 inhibitors, such as relatlimab; OX40 inhibitors; GITR inhibitors); CAR T-cell therapies (e.g., tisagenlecleucel, axicabtagene ciloleucel, brexucabtagene autoleucel, lisocabtagene maraleucel, idecabtagene vicleucel, ciltacabtagene autoleucel); monoclonal antibodies (e.g., alemtuzumab, trastuzumab, rituximab, blinatumomab, bevacizumab, cetuximab); vaccines; corticosteroids (e.g., prednisone, prednisolone, dexamethasone); disease-modifying antirheumatic drugs (DMARDs) (e.g., azathioprine, cyclosporine, hydroxychloroquine, leflunomide, methotrexate, sulfasalazine); cytokines (e.g., chemokines, such as interleukins, interferons, tumor necrosis factors, growth factors); Bacillus Calmette-Guerin (BCG); thalidomide; lenalidomide; pomalidomide; imiquimod; and TIM receptor agonists (e.g., an agonist described herein, such as in TIM Receptor Agonists).

Also provided herein, among other things, are methods of evaluating response of one or more non-human animals disclosed herein (e.g., a functional human immune system of one or more non-human animals disclosed herein) to a human pathogen (e.g., a virus, such as a virus that is not pathogenic in mice), comprising evaluating, in any one or more non-human animals disclosed herein, response the one or more non-human animals (e.g., the functional human immune system of the one or more non-human animals) to infection by the human pathogen. In some embodiments, the one or more non-human animals are infected with the human pathogen. In some embodiments, the one or more non-human animals are maintained under conditions sufficient for the human pathogen to infect the one or more non-human animals.

Also provided herein, among other things, are methods of evaluating response of one or more non-human animals disclosed herein (e.g., a functional human immune system of one or more non-human animals disclosed herein) to a human pathogen (e.g., a virus that is not pathogenic in mice), comprising:
exposing any one or more non-human animals disclosed herein to the human pathogen;
maintaining the one or more non-human animals under conditions sufficient for the human pathogen to infect the one or more non-human animals; and
evaluating the response of the one or more non-human animals (e.g., the functional human immune system of the one or more non-human animals) to infection by the human pathogens.

Also provided herein, among other things, are methods of evaluating an effect (e.g., safety and/or efficacy) of a vaccine or anti-infective (e.g., anti-viral agent) in one or more non-human animals disclosed herein, comprising administering the vaccine or anti-infective to the one or more non-human animals disclosed herein and evaluating the effect of the vaccine or anti-infective. In some embodiments, the one or more non-human animals are infected with a human pathogen (e.g., prior to receiving the vaccine or anti-infective; at the time of receiving the vaccine or anti-infective; after receiving the vaccine or anti-infective). In some embodiments, the one or more non-human animals are maintained under conditions sufficient for the human pathogen to infect the one or more non-human animals (e.g., prior to receiving the vaccine and/or after receiving the vaccine or anti-infective).

Also provided herein, among other things, are methods of evaluating an effect of a vaccine or anti-infective (e.g., anti-viral agent) in one or more non-human animal disclosed herein, comprising:
exposing any one or more non-human animals disclosed herein to a human pathogen or maintaining the one or more non-human animals under conditions sufficient for a human pathogen to infect the one or more non-human animals;
administering the vaccine or anti-infective to the one or more non-human animals; and
evaluating the effect of the vaccine or anti-infective.

In some embodiments, the human pathogen is a virus, for example, a virus selected from the group consisting of coronavirus (e.g., SARS, MERS or SARS-2 coronavirus), diphtheria, Ebola, hepatitis, human papillomavirus (HPV), immunodeficiency virus, influenza, morbillivirus, a norovirus, a paramyxovirus, poliovirus, respiratory syncytial virus (RSV), rotavirus, varicella zoster virus (VZV), lyssavirus, an enterovirus, variola virus, monkeypox virus, dengue virus (DENV), Japanese encephalitis virus (JEV), yellow fever virus (YFV), and Zika virus.

In some embodiments, the human pathogen is a bacterium selected from the group consisting of Bordetella pertussis, Clostridium tetani, Haemophilus influenzae type b, Neisseria meningitidis (meningococcus bacteria), Streptococcus pneumoniae, Salmonella typhi bacteria, Bacillus anthracis, Mycobacterium tuberculosis, and cholerae bacteria.

In some embodiments, a vaccine described herein is an infectious pathogen vaccine. In some embodiments, the infectious pathogen vaccine is an anti-viral vaccine. In some embodiments, an antiviral vaccine described herein is a COVID-19 vaccine, an influenza vaccine, a diphtheria vaccine, an Ebola vaccine, a hepatitis (e.g., hepatitis A or hepatitis B) vaccine, an HIV/AIDS vaccine, a human papillomavirus (HPV) vaccine, a measles vaccine, a mumps vaccine, a polio vaccine, a rotavirus vaccine, a rubella vaccine, a varicella-zoster virus (VZV) vaccine, a shingles vaccine, a rabies vaccine, a smallpox vaccine, a monkeypox vaccine, an adenovirus vaccine, a dengue vaccine, a Japanese encephalitis virus (JEV) vaccine, or a yellow fever virus (YFV) vaccine.

### EXEMPLIFICATION

### Example 1. Generating a Mouse Comprising a Functional Human Immune System

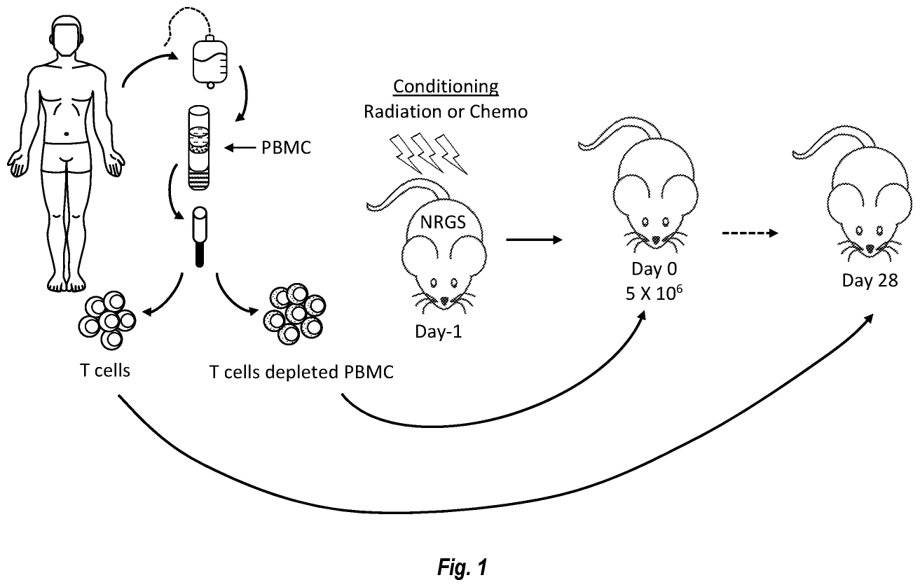

Mice comprising a functional human immune system were generated according to the workflow depicted in FIG. 1.

### 1A. Animals

NRGS mice (also known as NRG-SGM3 or NRG-3GS) were obtained from the Jackson Laboratory (Bar Harbor, ME) and maintained and bred at an animal facility at Tufts University per Institutional Animal Care and Use Committee (IACUC). The NRGS mice are NOD.Rag1^{-/-}; γc^{null} (NRG) animals expressing human interleukin-3 (IL-3), human granulocyte/macrophage-stimulating factor (GM-CSF) and human Steel factor (SF) from the SGM3 (3GS) triple co-injected transgenes (jax.org/strain/024099). Human IL-3 and GM-CSF are important for myeloid cell development, and Human SF helps with engraftment. Mice were conditioned with busulfan (250 mg/kg, sub-lethal dose) a day before engrafting peripheral blood mononuclear cells (PBMCs).

### 1B. Peripheral Blood Mononuclear Cell Preparation, Freezing & Shipping

Before commencing the preparation, all reagents and the centrifuge were at room temperature.

TABLE 1
Materials Needed for PBMC Preparation
from a Whole Blood Sample

| Material | Provider | Catalog No. |
|---|---|---|
| 15 mL Tubes - Sterile | US Plastic | 76938 |
| Tube, 50 mL, Polypropylene, Sterile | US Plastic | 76941 |
| 25 mL Serological Pipet | Drummond | 6-000-025 |
| Ficoll | GE Healthcare | 17144002 |
| Fetal Bovine Serum (FBS) | Gibco | 26400044 |
| Phosphate Buffered Saline (PBS) 1x | Gibco | 10010049 |
| Cryovial | Fisher Scientific | 09-761-70 |
| Isopropanol Freezing Container | Thermo Fisher | 5100-0001 |
| DMSO | Thermo Fisher | TS-20688 |

### Preparing PBMCs

1. Added PBS (at room temperature) to a blood sample from a healthy human at a 1:1 volume-to-volume ratio.
2. In an appropriate sized tube, added a volume of Ficoll that was equal to ½ (one half) volume of the diluted blood from step 1.
3. Slowly and gently loaded the diluted blood from step 1 to the Ficoll from step 2 using 25 mL serological pipet without mixing the blood with Ficoll, to yield a 2:1 volume-to-volume ratio.
4. Centrifuged the tube from step 3 at 700 g (see e.g., a relative centrifugal force (RCF) to revolutions per minute (RPM) conversion table from Sigma Aldrich (sigmaaldrich.com/US/en/support/calculators-and-apps/g-force-calculator). for RCF to RPM conversion), room temperature for 30 minutes, with minimal acceleration and without break.
5. After the centrifugation, the blood components were separated into four layers.
6. Gently inserted the tip of a pipet through the plasma layer into the PBMC layer and collected the PBMCs.
7. Transferred the collected PBMCs into a new 50 ml tube.
8. Filled the tube from step 7 with PBS.
9. Centrifuged the tube from step 8 at 360 g, 4° C. for 10 minutes. Discarded the supernatant and resuspending the pellet with 50 ml cold PBS.
10. Centrifuged the tube from step 9 at 120 g, 4° C. for 10 minutes. Gently aspirated the supernatant without moving the cell pellet.
11. Resuspended the cells from step 10 in 30 ml of PBS.
12. Counted the cells as follows:
   a. Added 10 μl of the cell suspension from step 11 to 90 μl of trypan blue;
   b. Mixed well, and loaded 10 μl of the mixture from step “a” into a LUNA™ cell counting slide;
   c. Turned on the LUNA-II™ automated cell counter;
   d. Inserted the slide into the cell counter; and
   e. Calculated the total cell number by multiplying the count from the LUNA-II™ automated cell counter by 30,000 (the dilution factor).
13. Adjusting the cell concentration using FBS to obtain 20-40×10⁶ cells/ml FBS, and keeping the cells on ice or 4° C. (e.g., until freezing).

### Freezing PBMCs

### Before Starting:

Pre-chilled labeled cryovials and an isopropanol freezing container at 4° C.;
Prepared a sufficient amount of 20% dimethyl sulfoxide (DMSO) in FBS based on the total volume obtained in step 13.

### Procedure:

Added 0.5 ml of the 20% DMSO-FBS solution to each cryovial.
Added 0.5 ml of the cells to each cryovial, and mixed the cells with the DMSO-FBS solution.
Placed the cryovials in the isopropanol freezing container at −80° C. for 24 hours, followed by transferring the vials into liquid nitrogen.

### 1C. Depleting T Cells from the Peripheral Blood Mononuclear Cells

Magnetic Labeling. To deplete T cells from the PBMCs, the CD3⁺ cells were magnetically labeled with CD3 MicroBeads (Miltenyi Biotec, Bergisch Gladbach, Germany, No. 130-050-101) according to the manufacturer's protocols (www.miltenyibiotec.com):
1. Determined cell number.
2. Centrifuged cell suspension at 300 g for 10 minutes. Pipetted off supernatant completely.
3. Resuspended the cell pellet in 80 μl of buffer per 10⁷ total cells.
4. Added 20 μl of CD3 MicroBeads per 10⁷ total cells.
5. Mixed well, and incubated at 4-8° C. for 15 minutes.
6. (Optional) Added staining antibodies (e.g., added 10 μl of CD3-FITC (e.g., Miltenyi Biotec, Bergisch Gladbach, Germany, No. 130-080-401), and incubated at 4-8° C. for 5 minutes.
7. Washed cells by adding 1-2 mL of buffer per 10⁷ cells, centrifuged at 300 g for 10 minutes, and pipetted off supernatant completely.
8. Resuspended up to 10⁸ cells in 500 μl of buffer.

Magnetic Separation. The cell suspension was loaded onto a magnetic-activated cell sorting (MACS®) LS column placed in a magnetic field of a MACS separator according to the manufacturer's protocols (www.miltenyibiotec.com):
1. Placed an LS column in the magnetic field of a suitable MACS Separator (“Column data sheets”).
2. Prepared the LS column by rinsing with an appropriate amount of buffer (3 ml).
3. Applied the cell suspension onto the LS column.
4. Collected unlabeled cells (which pass through) and washed the column with an appropriate amount of buffer. Performed washing steps by adding 3 ml buffer three times, each time once the column reservoir is empty. Collected total effluent, which was the unlabeled cell fraction.
5. Removed the column from the separator and placed it on a suitable collection tube.
6. Pipetted an appropriate amount (5 ml) of buffer onto the LS column. Immediately flushed out fraction with the magnetically labeled cells by firmly applying the plunger supplied with the column.

Briefly, a population of 100×10⁶ PBMCs or leukocytes were resuspended in 900 μl of MACS buffer. 100 μl of CD3 microbeads were added, and the cells were incubated at 4° C. (or on ice) for 30 minutes. The cells were washed with MACS buffer and centrifuged at 360 g, 4° C. for 5 minutes. Then the cells were resuspended in 5 ml of MACS buffer and loaded over a MACS buffer-prewashed LS column (Miltenyi Biotec, Bergisch Gladbach, Germany). After all cells ran through the column, two washes of 3 ml of MACS buffer were applied. The negative fraction (flow-through) was collected as T-cell depleted PBMCs. The positive fraction comprised T cells labeled with the CD3 microbeads.

### 1D. Preparing Lymphocytes

The lymphocytes implanted on Day 28 in accordance with the workflow depicted in FIG. 1 comprised CD4⁺ T cells and CD19⁺ B cells. A population of 100×10⁶ PBMC or leukocytes were resuspended in 900 μl of MACS buffer, incubated with 100 μl of either CD4 microbeads for CD4⁺ T cell selection, or CD19⁺ microbeads for CD19⁺ B cell selection, at 4° C. (or on ice) for 30 minutes. The cells were washed with MACS buffer and centrifuge at 360 g, 4° C. for 5 minutes. Then the cells were resuspended in 5 ml MACS buffer and loaded over a prewashed MACS LS column. The positive fraction comprised the CD4⁺ T cells (labeled with the CD4 microbeads) or CD19⁺ B cells (labeled with the CD19 microbeads).

In some experiments, lymphocytes implanted on Day 28 comprised CD4⁺ T cells, but lacked CD19⁺ B cells, and further lymphocytes were implanted on Day 56 (not shown in FIG. 1). Lymphocytes implanted on Day 56 comprised CD8⁺ T cells from the same human donor which supplied the CD4⁺ T cells and PBMCs (containing a population of CD19⁺ B cells). In these experiments, PBMCs (containing a population of CD19⁺ B cells) were implanted on Day 0, CD4⁺ T cells were implanted on Day 28 and CD8⁺ T cells were implanted on Day 56. The human CD8⁺ T cells were separated magnetically from frozen PBMCs using a CD8⁺ T Cell Isolation Kit (Miltenyi Biotec, Bergisch Gladbach, German). PBMCs from human donors were thawed and washed with MACS buffer (PBS containing 2% FBS and 1 mM EDTA). Cells were counted, and 100 μl CD8 isolating microbeads were added for every 100×10⁶ per 1 ml of MACS buffer. Cells were incubated with beads for 20 minutes on ice, then washed and rung through the isolation MS column from the kit. The column was washed three times with MACS buffer. The positive fraction was flushed by 5 ml of MACS in a 15-ml tube, the cell suspension was counted, spun down, and the concentration was adjusted to 5×10⁶ cells/ml. 100 μl of the CD8⁺ T cells were intravenously injected into one or more mice that had previously received the same donor's T-cell depleted PBMCs and the same donor's CD4⁺ T cells.

### 1E. Cell Graft

In the workflow depicted in FIG. 1, on Day 0 (about 24 hours post-conditioning), the preconditioned mice received 5×10⁶ T-cell depleted PBMCs or 10×10⁶ T cell-depleted leukocytes via intravenous (i.v.) injection. On Day 28, the mice received 1×10⁶ CD4⁺ T cells, together with 0.5×10⁶ CD19⁺ B cells.

In mice receiving CD8⁺ T cells, on Day 0 (about 24 hours post-conditioning), the preconditioned mice received T-cell depleted PBMC via i.v. injection. On Day 28, the mice received CD4⁺ T cells. On Day 56, the mice received 0.5×10⁶ CD8⁺ T cells.

### Example 2. Characterization of Engrafted Mice from Example 1

The immune systems of 14 human donors were engrafted into a total of 60 conditioned mice using the procedure described in Example 1, with 100% viability at the end of the engraftment period (e.g., Day 28 or Day 56). To date (6 months post engraftment). The engrafted mice showed no sign of xenorejection as described in Ramadan, A. M., et al., JCI Insight 2018; 3(14):e99208. The engrafted mice generated were chimeric with respect to hematopoietic cells (as indicated by the presence of the hematopoietic cell marker, CD45). However, these mice had exclusively human T cells and human B cells.

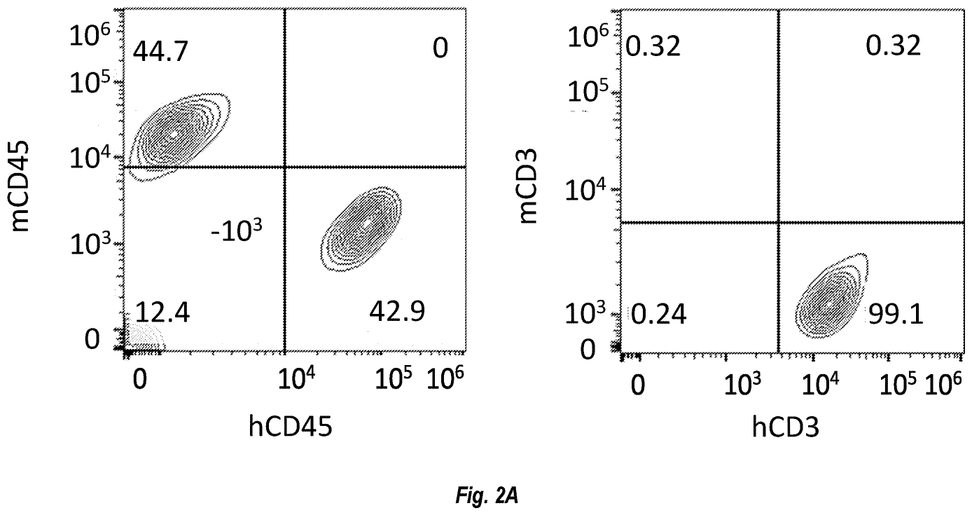

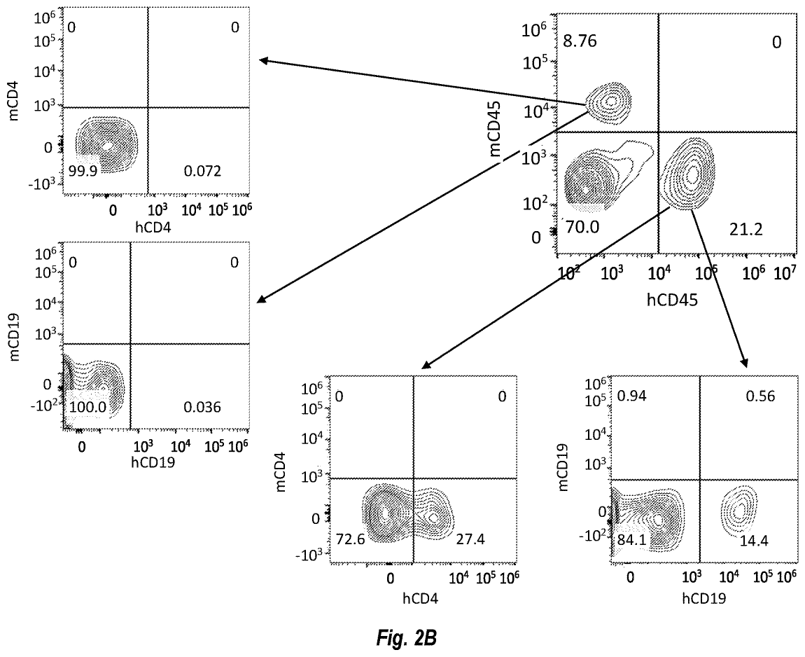

The immune phenotype of the engrafted mice was evaluated using fluorescence-activated single cell sorting (FACS) (FIGS. 2A-2B). As shown in FIG. 2A, while expression of both mouse and human CD45 was observed in T cells, only human CD3 was observed in T cells of the engrafted mice. As shown in FIG. 2B, while both mouse CD45⁺ and human CD45⁺ cells were detected, mouse CD45⁺ cells were negative for CD4 and negative for CD19, indicating a lack of mouse T cells and a lack of mouse B cells in the engrafted mice. In contrast, both human CD4 and human CD19 were detected in human CD45⁺ cells in the engrafted mice.

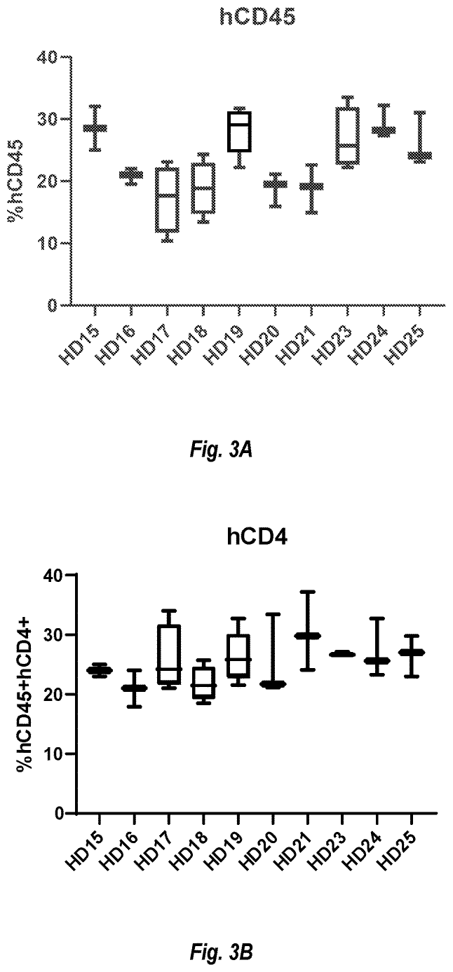

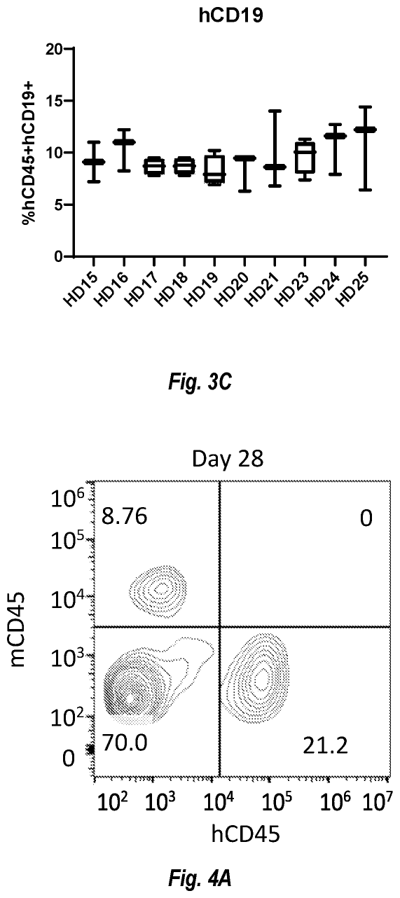

FIGS. 3A-3C shows that reconstitution of human immune cells, as described in the engraftment protocol of Example 1, is highly reproducible. Of 34 mice engrafted with cells from 10 healthy human donors, the average percentages of human CD45⁺ cells, human CD45⁺/CD4⁺ T cells, and human CD45⁺/CD19⁺ B cells were 23.4% (17.2-29.2%, FIG. 3A), 25.5% (21.0-30.4%, FIG. 3B), and 9.5% (8.3-11%, FIG. 3C), respectively.

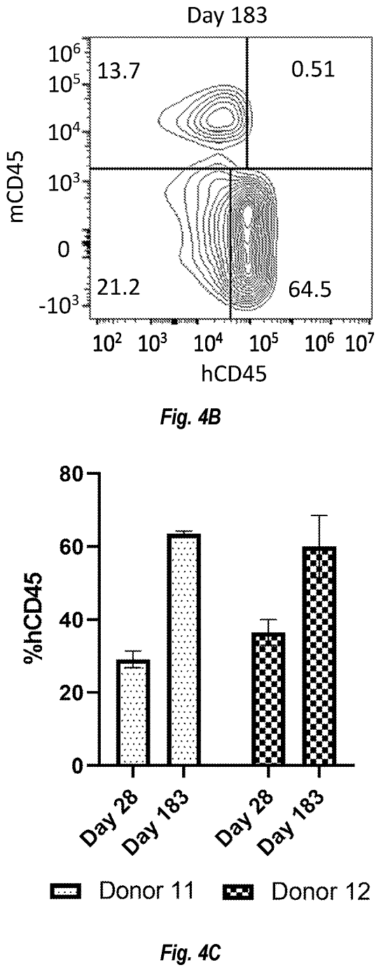

FIGS. 4A-6C show the stability of engrafted mice and duration of engraftment. FIG. 4A shows expansion of human hematopoietic cells over time in the engrafted mice. In four mice engrafted with cells from two human donors, the percentage of human hematopoietic (CD45⁺) cells increased from 32% (SD=5.2%) on day 28 to 61.7% (SD=2.4%) on day 183 (FIGS. 4A-4C).

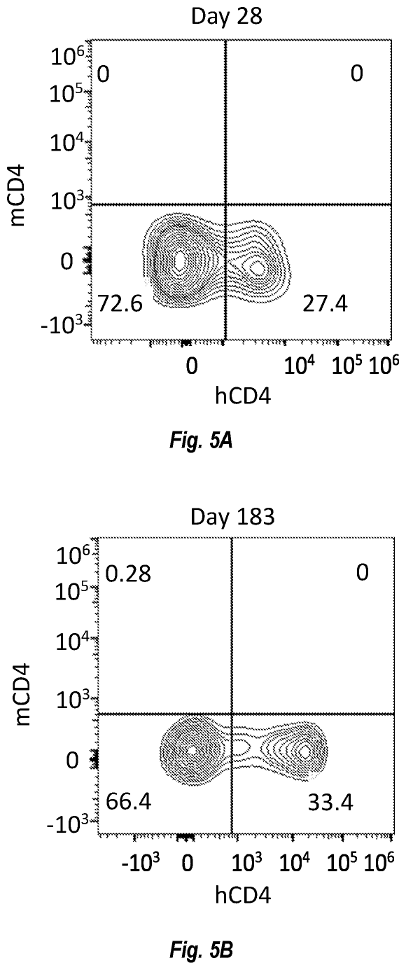

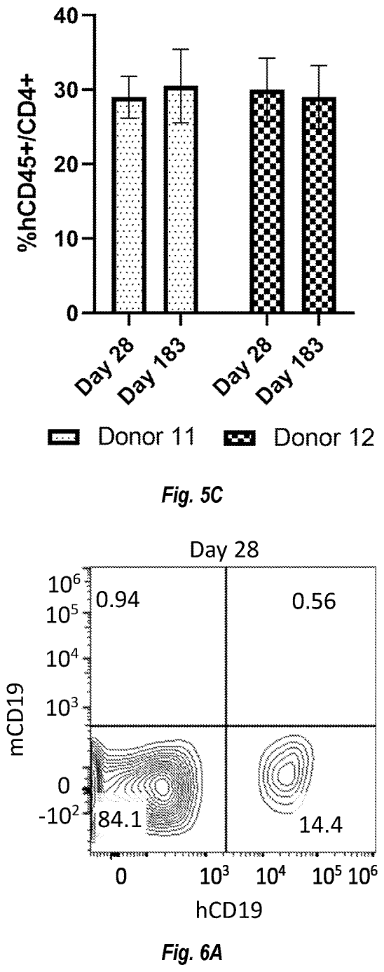

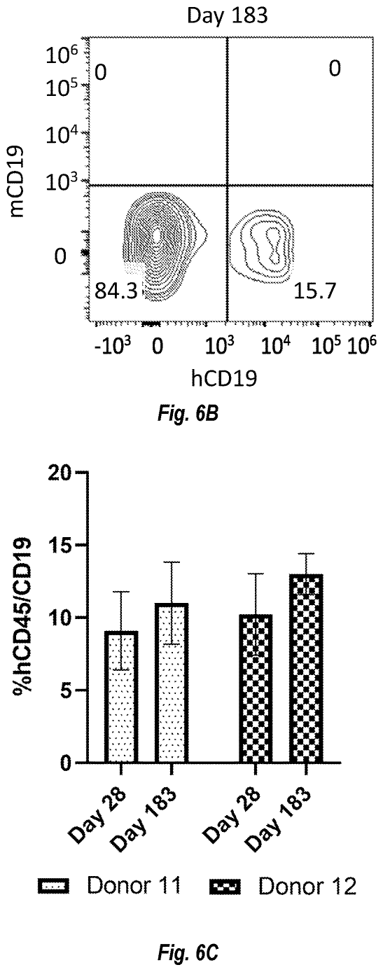

Engrafted mice are also highly stable with respect to hCD4 (T cell) and hCD19 (B cell) markers, and to the ratio of T cells to B cells. The percentage of human CD4⁺ T cells in the human hematopoietic cells was maintained between days 28 (29.5%) and 183 (29.7%) (FIGS. 5A-5C); and the percentage of human CD19⁺ B cells in the human hematopoietic cells was maintained between days 28 (9.6%) and 183 (12%) (FIGS. 6A-6C).

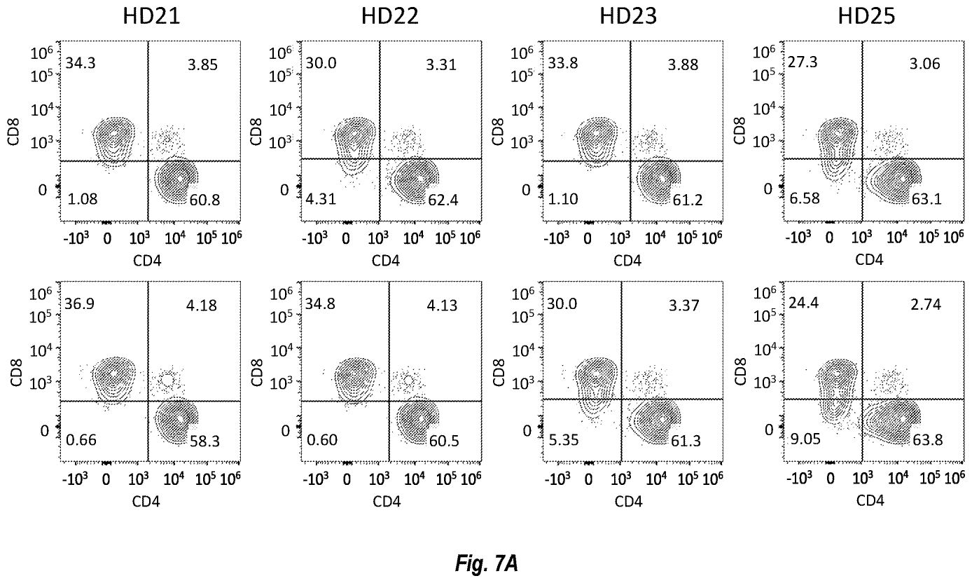

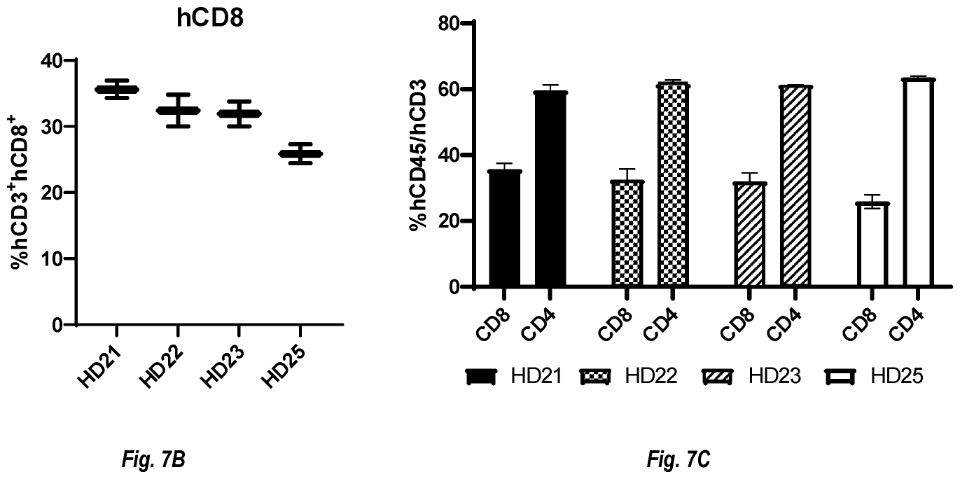

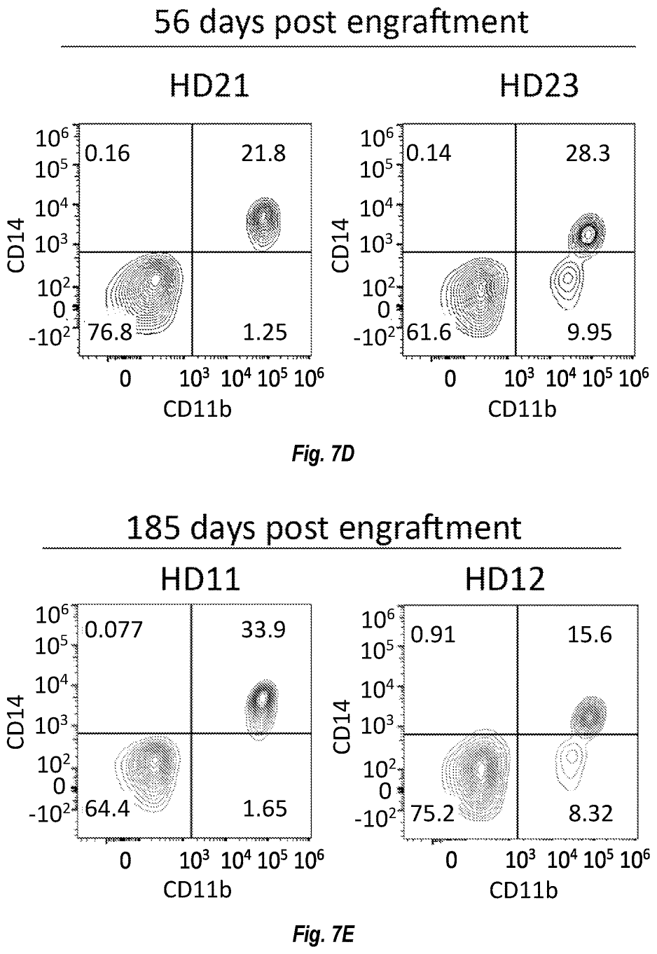

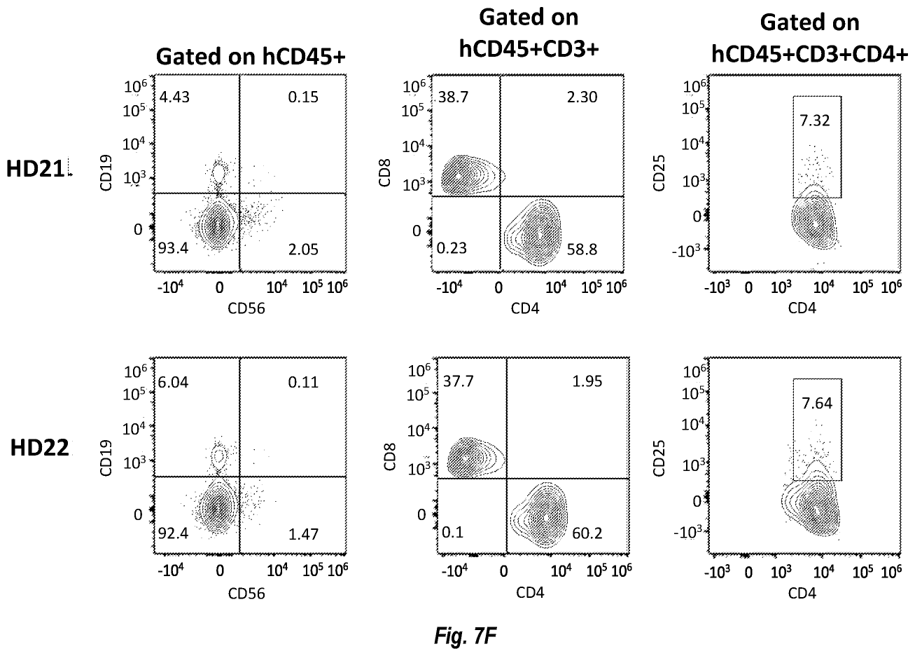

Absence of graft-versus-host disease (xenorejection) has not been reported in mouse models for engraftment of human CD8⁺ cells from healthy or diseased human donors (e.g., a cancer patient donor). However, engrafted mice generated in accordance with the procedures described herein were viable and had no signs of xenorejection. Furthermore, the ratio of CD8⁺ T cells to CD4⁺ T cells (about 1:3) was maintained in the engrafted mice, and was approximately physiological (FIGS. 7A-7C).

Engrafted mice have persistent human myeloid cells for antigen presentation and persistent human NK cells. CD14⁺/CD11b⁺ cells are cells of myeloid lineage. Myeloid cells may comprise monocytes, macrophages, myeloid dendritic cells (mDCs), granulocytes, and/or mast cells, and originate from a common myeloid progenitor in the bone marrow. The percentage of CD14⁺/CD11b⁺ cells in mice engrafted with cells from HD21 and HD23 was 21.8% and 28.3%, respectively, 56 days post-engraftment (FIG. 7D). The percentage of CD14⁺/CD11b⁺ cells in mice engrafted with cells from HD11 and HD12 was 33.9% and 15.6%, respectively, 185 days post-engraftment (FIG. 7E).

Engrafted mice also have persistent human NK cells. CD3 is a marker for T cells; CD4 is a marker for Tₕₑₗₚₑᵣ cells; CD8 is a marker for cytotoxic T cells; CD19 is a marker for B cells; CD25 is a marker for activated T cells/T_{regs}; and CD56 is a marker for NK cells. Seven months after protocol initiation, mice engrafted with cells from HD21 and HD22 had 2.05% and 1.47% human NK cells, respectively (FIG. 7F, leftmost plots). FIG. 7F also shows that seven months after protocol initiation, engrafted mice engrafted with cells from HD21 and HD22 continued to have a physiological and healthy ratio of CD8⁺ T cells to CD4⁺ T cells (FIG. 7F, middle plots), and a normal amount of activated T cells/T_{regs} (FIG. 7F, rightmost plots, 7.32% and 7.64%, respectively).

The data suggest that the engrafted mice can be used as models for studying autologous and allogenic cancers (e.g., hematological cancers or solid tumors).

### Example 3. Predicting Human Immunogenicity

### 3A. Immunogenicity Against FVIII

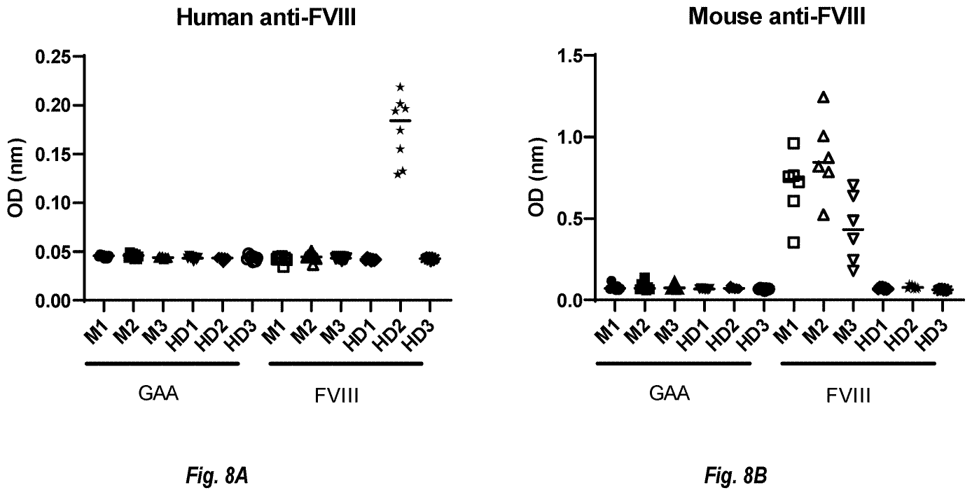

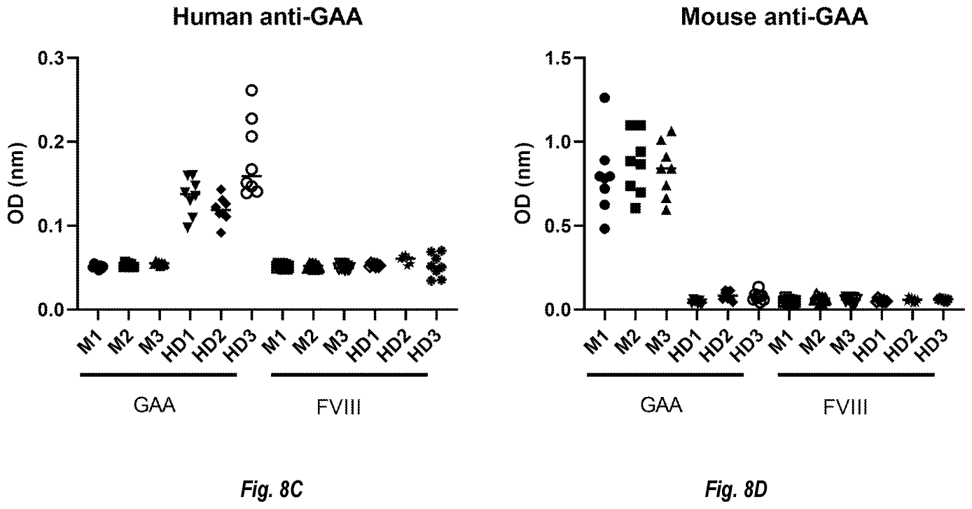

Three healthy human donors were used to generate three engrafted mice designated HD1, HD2 and HD3. On Day 29 and Day 36, HD1, HD2 and HD3 were administered FVIII. The same amounts of antigen were administered to three C57B/6 control mice on the same date. Two weeks later (on Day 43), blood from each mouse was collected. Titers of mouse anti-FVIII antibodies, human anti-FVIII antibodies, mouse anti-GAA antibodies and human anti-GAA antibodies were measured using ELISA (FIGS. 8A-8B).

As shown in FIG. 8A, one out of the three engrafted mice (H1D2) administered FVIII developed human anti-FVIII antibodies. None of the C57B/6 control mice or engrafted mice administered GAA developed human anti-FVIII antibody titer (FIG. 8A), indicating that the FVIII titer assay was specific. The observed immunogenicity rate in the engrafted mice, about 33%, is consistent with the clinically reported immunogenicity rate to FVIII in humans (about 30%).

These experiments were expanded to include additional mice. The results were consistent when the number of mice was increased: 25% of 12 engrafted mice administered FVIII developed human anti-FVIII antibodies; and 100% of six C57B/6 mice administered FVIII developed mouse anti-FVIII antibodies.

As shown in FIG. 8B, none of the engrafted mice administered FVIII developed mouse anti-FVIII antibodies. In contrast, all three (100%) C57B/6 mice administered FVIII developed mouse anti-FVIII anti-bodies (FIG. 8B). Finally, none of the C57B/6 control mice or engrafted mice administered GAA developed mouse anti-FVIII antibody titer (FIG. 8B).

### 3B. Immunogenicity Against GAA

Three healthy human donors were used to generate three engrafted mice designated HD1, HD2 and HD3. On Day 29 and Day 36, HD1, HD2 and HD3 were administered GAA. The same amounts of antigen were administered to three C57B/6 control mice on the same date. Two weeks later (on Day 43), blood from each mouse was collected. Titers of mouse anti-GAA antibodies, human anti-GAA antibodies, mouse anti-FVIII antibodies and human anti-FVIII antibodies were measured using ELISA (FIGS. 8C-8D).

As shown in FIG. 8C, all three engrafted mice administered GAA developed human anti-GAA antibodies. None of the C57B/6 control mice or engrafted mice administered FVIII developed human anti-GAA antibody titer (FIG. 8C), indicating that the GAA titer assay was specific. The observed immunogenicity rate in the engrafted mice, about 100%, is consistent with the clinically reported immunogenicity rate to GAA in humans (about 89-100%).

This experiment was expanded to include additional mice. The results were consistent when the number of mice was increased: 100% of 14 engrafted mice administered GAA developed human anti-GAA antibodies; and 100% of 5 C57B/6 mice administered GAA developed mouse anti-GAA antibodies.

As shown in FIG. 8D, none of the engrafted mice administered GAA developed mouse anti-GAA antibodies. In contrast, all three (100%) C57B/6 mice administered GAA developed mouse anti-GAA anti-bodies (FIG. 8D). Finally, none of the C57B/6 control mice or engrafted mice administered FVIII developed mouse anti-GAA antibody titer (FIG. 8D).

### 3C. Immunogenicity Against IFN-β

Healthy human donors were used to generate eight engrafted mice. On Day 29 and Day 36, the engrafted mice were administered IFN-β. The same amount of antigen was administered to three C57B/6 control mice on the same date. Two weeks later (on Day 43), blood from each mouse was collected. Titers of mouse anti-IFN-β antibodies and human anti-IFN-β antibodies were measured using ELISA.

25% of the eight engrafted mice administered IFN-β developed human anti-IFN-β antibodies; and 100% of the three C57B/6 mice administered IFN-β developed mouse anti-IFN-β antibodies. The observed immunogenicity rate in the engrafted mice is consistent with the clinically reported immunogenicity rate to IFN-β in humans (about 28-47%). None of the engrafted mice administered IFN-β developed mouse anti-IFN-β antibodies.

### 3D. Immunogenicity Against AAV9

Healthy human donors were used to generate 11 engrafted mice. On Day 29 and Day 36, the engrafted mice were administered AAV9. The same amount of antigen was administered to three C57B/6 control mice on the same date. Two weeks later (on Day 43), blood from each mouse was collected. Titers of mouse anti-AAV9 antibodies and human anti-AAV9 antibodies were measured using ELISA.

100% of the 11 engrafted mice administered AAV9 developed human anti-AAV9 antibodies; and 100% of the three C57B/6 mice administered AAV9 developed mouse anti-AAV9 antibodies. The observed immunogenicity rate in the engrafted mice is consistent with the clinically reported immunogenicity rate to AAV9 in humans (about 100%). None of the engrafted mice administered AAV9 developed mouse anti-AAV9 antibodies.

Presence of human CD20 was detected on B cells of mice that developed antibodies to different biologics. CD20 is expressed on activated, antibody-producing B cells. FIGS. 9A-9C show that engrafted mice exposed to FVIII, GAA, or AAV9, respectively, had CD20-expressing B cells, consistent with B cell maturation induced by the FVIII, GAA, or AAV9.

These data demonstrate that the NRGS mouse model can predict the clinical immunogenicity rate for biologics.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A non-human animal, wherein the non-human animal:
a) lacks endogenous mature T cells; and
b) comprises an engrafted population of human mononuclear cells, human polymorphonuclear leukocytes, or both, wherein the engraftment lacks human cancer cells.

2. A non-human animal, wherein the non-human animal:
a) lacks endogenous mature T cells; and
b) comprises a functional human immune system generated from an engraftment of a population of human mononuclear cells, human polymorphonuclear leukocytes, or both, wherein the engraftment lacks human cancer cells.

3. A method of generating a non-human animal, comprising engrafting a population of human mononuclear cells, human polymorphonuclear leukocytes, or both into a non-human animal lacking endogenous mature T cells, wherein the engraftment lacks human cancer cells.

4. A method of generating a non-human animal, comprising engrafting a population of human mononuclear cells, human polymorphonuclear leukocytes, or both into a non-human animal lacking endogenous mature T cells, wherein the engraftment lacks human cancer cells, and wherein the engraftment produces a functional human immune system in the non-human animal.

5. The non-human animal of claim or the method of any one of claims 1-4, wherein the non-human animal lacks endogenous mature B cells, endogenous mature NK cells, or both.

6. The non-human animal or the method any one of claims 1-5, wherein the non-human animal comprises one or more human lymphocyte cell types, one or more human myeloid cell types, or any combination thereof.

7. The non-human animal or the method claim 6, wherein the one or more human lymphocyte cell types comprise human T cells, human B cells, human NK cells, or any combination thereof.

8. The non-human animal or the method claim 6, wherein the one or more human myeloid cell types comprise human monocytes, human macrophages, or both.

9. The non-human animal or the method any one of claims 1-8, wherein the non-human animal comprises human T cells, human B cells, human NK cells, human monocytes, and human macrophages.

10. The non-human animal or the method any one of claims 1-9, wherein the non-human animal comprises both endogenous CD45+ cells and human CD45+ cells.

11. The non-human animal or the method any one of claims 7-10, wherein the human T cells comprise human CD4+ T cells, human CD8+ T cells, or both.

12. The non-human animal or the method any one of claims 1-11, wherein CD4⁺ T cells in the non-human animal comprise only human CD4⁺ T cells, wherein CD8+ T cells in the non-human animal comprise only human CD8⁺ T cells, or both.

13. The non-human animal or the method any one of claims 7-12, wherein the human B cells comprise human CD19+ B cells.

14. The non-human animal or the method any one of claims 7-13, wherein the human NK cells comprise human CD56+ NK cells.

15. The non-human animal or the method any one of claims 6-14, wherein the one or more human myeloid cell types comprises CD14+/CD11b+ cells.

16. The non-human animal or the method of any one of claims 1-15, wherein the non-human animal is a rodent.

17. The non-human animal or the method of claim 16, wherein the rodent is a mouse.

18. The non-human animal or the method of any one of claims 1-17, wherein the non-human animal lacks endogenous mature B cells.

19. The non-human animal or the method of any one of claims 1-18, wherein the non-human animal lacks endogenous mature natural killer (NK) cells.

20. The non-human animal or the method of any one of claims 1-19, wherein less than 15%, less than 10%, less than 5%, or less than 1% of CD3 cells in the non-human animal are endogenous CD3 cells.

21. The non-human animal or the method of any one of claims 1-20, wherein the non-human animal:
a) expresses human interleukin-3 (IL-3);
b) expresses human granulocyte/macrophage-stimulating factor (GM-CSF);
c) expresses human steel factor (SF);
d) lacks recombination activating 1 (Rag1) expression, function, or both;
e) lacks IL2 receptor common gamma chain (IL2rγ) expression, function, or both;
f) lacks recombination activating 2 (Rag2) expression, function, or both;
g) lacks DNA-dependent protein kinase (DNA-PK) expression, function, or both;
h) lacks β2 microglobulin (B2m) expression, function, or both; or
i) lacks perforin (Prf1) expression, function, or both;
j) lacks T-cell receptor (TCR) α chain expression, function, or both; or
k) lacks major histocompatibility complex (MHC) class II expression, function, or both; or
l) any combination of (a)-(k).

22. The non-human animal or the method of any one of claims 1-21, wherein the non-human animal is a NRGS mouse, a NSGS mouse, a huNOG mouse, or a scid mouse.

23. The non-human animal or the method of claim 21 or 22, wherein the non-human animal:
a) expresses human IL-3;
b) expresses human GM-CSF;
c) expresses human SF;
d) lacks Rag1 expression, function, or both; and
e) lacks IL2rγ expression, function, or both.

24. The non-human animal or the method of any one of claims 1-23, wherein the non-human animal expresses human interleukin-5 (IL-5), human interleukin-7 (IL-7), or both.

25. The non-human animal or the method of any one of claims 1-24, wherein the non-human animal is conditioned with radiation, chemotherapy, or a combination thereof.

26. The non-human animal or the method of any one of claims 1-25, wherein the non-human animal is conditioned with radiation.

27. The non-human animal or the method of any one of claims 1-26, wherein the non-human animal is conditioned with radiation at a dose of about 150 cGy to about 300 cGy.

28. The non-human animal or the method of any one of claims 1-27, wherein the non-human animal is a NRGS mouse.

29. The non-human animal or the method of claim 28, wherein the NRGS mouse is conditioned with radiation at a dose of about 300 cGy.

30. The non-human animal or the method of any one of claims 1-27, wherein the non-human animal is a NSGS mouse.

31. The non-human animal or the method of claim 30, wherein the NSGS mouse is conditioned with radiation at a dose of about 150 cGy.

32. The non-human animal or the method of any one of claims 1-31, wherein the non-human animal is conditioned with chemotherapy.

33. The non-human animal or the method of claim 32, wherein the chemotherapy comprises busulfan, cyclophosphamide, or cyclosporine, or any combination of the foregoing.

34. The non-human animal or the method of any one of claims 1-33, wherein the human mononuclear cells, the human polymorphonuclear leukocytes, or both, are from at least one human.

35. The non-human animal or the method of any one of claims 1-34, wherein the human mononuclear cells, the human polymorphonuclear leukocytes, or both, are from two or more humans.

36. The non-human animal or the method of claim 34, wherein the human mononuclear cells and the human polymorphonuclear leukocytes are from at least one human.

37. The non-human animal or the method of claim 34, wherein the human mononuclear cells and the human polymorphonuclear leukocytes are from two or more humans.

38. The non-human animal or the method of any one of claims 1-37, wherein the at least one human is a healthy human.

39. The non-human animal or the method of any one of claims 1-38, wherein the at least one human is about 6 months to about 17 years of age or is about 18 years or older.

40. The non-human animal or the method of any one of claims 1-39, wherein the at least one human received a treatment to mobilize stem cells.

41. The non-human animal or the method of claim 40, wherein the treatment to mobilize stem cells comprises a granulocyte colony-stimulating factor (G-CSF).

42. The non-human animal or the method of any one of claims 1-41, wherein the population of human mononuclear cells, human polymorphonuclear leukocytes, or both is a population of human mononuclear cells and human polymorphonuclear leukocytes.

43. The non-human animal or the method of any one of claims 1-42, wherein the population of human mononuclear cells, human polymorphonuclear leukocytes, or both is a population of human mononuclear cells.

44. The non-human animal or the method of any one of claims 1-43, wherein the population of human mononuclear cells comprises peripheral blood mononuclear cells (PBMCs), umbilical cord blood mononuclear cells (CB-MNCs), bone marrow mononuclear cells (BMNCs), or a combination thereof.

45. The non-human animal or the method of any one of claims 1-44, wherein the population of human mononuclear cells comprises peripheral blood mononuclear cells (PBMCs).

46. The non-human animal or the method of any one of claims 1-45, wherein the population of human mononuclear cells comprises human T cells, human B cells, human monocytes, human macrophages, and human NK cells.

47. The non-human animal or the method of any one of claims 1-46, wherein the population of human mononuclear cells comprises about 5×10² to about 1×10⁹ human mononuclear cells.

48. The non-human animal or the method of any one of claims 1-42, wherein the population of human mononuclear cells, human polymorphonuclear leukocytes, or both is a population of human polymorphonuclear leukocytes.

49. The non-human animal or the method of any one of claims 1-42 and 48, wherein the population of human polymorphonuclear leukocytes comprises peripheral blood polymorphonuclear leukocytes, umbilical cord blood polymorphonuclear leukocytes, bone marrow polymorphonuclear leukocytes, or a combination thereof.

50. The non-human animal or the method of any one of claims 1-49, wherein the population of human mononuclear cells, human polymorphonuclear leukocytes, or both is T-cell depleted.

51. The method of any one of claims 4-50, wherein a functional human immune system is generated from an engraftment of the population of human mononuclear cells, human polymorphonuclear leukocytes, or both and an engraftment of a population of human lymphocytes thereafter.

52. The method of claim 51, wherein the population of human lymphocytes is engrafted about 7-28 days after engrafting the population of human mononuclear cells, human polymorphonuclear leukocytes, or both into the non-human animal.

53. The method of claim 51 or 52, wherein the population of human lymphocytes is engrafted about 28 days after engrafting the population of human mononuclear cells, human polymorphonuclear leukocytes, or both into the non-human animal.

54. The method of any one of claims 51-53, wherein the population of human lymphocytes comprises a population of human CD4⁺ T cells, human CD8⁺ T cells, human CD4⁺ T cells and human CD8⁺ T cells, or total human T cells.

55. The method of any one of claims 51-54, wherein the population of human lymphocytes comprises a population of human CD4⁺ T cells.

56. The method of any one of claims 51-55, wherein the population of human lymphocytes comprises about 1×10⁶ human CD4⁺ T cells.

57. The method of any one of claims 51-56, wherein the population of human lymphocytes comprises a population of human B cells.

58. The method of claim 57, wherein the population of human B cells comprises human CD19⁺ naïve B cells, human memory B cells, human plasma cells, or a combination thereof.

59. The method of any one of claims 51-58, wherein the population of human lymphocytes comprises about 5×10⁵ human CD19⁺ B cells.

60. The non-human animal or the method of any one of claims 1-49 and 51-59, wherein the population of human mononuclear cells, human polymorphonuclear leukocytes, or both is not T-cell depleted.

61. The non-human animal or the method of any one of claims 1-60, wherein a ratio of mature human T cells to mature human B cells in the non-human animal is approximately equivalent to that found in healthy humans.

62. The non-human animal or the method of any one of claims 1-60, wherein a ratio of mature human T cells to mature human B cells in the non-human animal is approximately equivalent to that found in the at least one human.

63. The non-human animal or the method of any one of claims 1-60, wherein a ratio of mature human T cells to mature human B cells in the non-human animal is between about 1.5 and 6, between about 1.5 and 5, between about 1.5 and 4.5, between about 1.5 and 4, between about 1.5 and 3.5, between about 1.5 and 3, between about 1.5 and 2.5, between about 2 and 6, between about 2 and 5, between about 2 and 4, or between about 2 and 3.

64. The non-human animal or the method of any one of claims 1-63, wherein a ratio of human CD8⁺ T cells to human CD4⁺ T cells in the non-human animal is approximately equivalent to that found in healthy humans.

65. The non-human animal or the method of any one of claims 1-63, wherein a ratio of human CD8⁺ T cells to human CD4⁺ T cells in the non-human animal is approximately equivalent to that found in the at least one human.

66. The non-human animal or the method of any one of claims 1-63, wherein a ratio of human CD8⁺ T cells to human CD4⁺ T cells in the non-human animal is between about 1 and 4, such as between about 1 and 3.75, between about 1 and 3.5, between about 1 and 3.25, between about 1 and 3, between about 1 and 2.75, between about 1 and 2.5, between about 1 and 2.25, between about 1 and 2, between about 1 and 1.75, between about 1 and 1.5, or between about 1 and 1.25.

67. The method of any one of claims 3-66, further comprising administering a TIM receptor agonist to the non-human animal.

68. The method of any one of claims 3-662, further comprising incubating the population of human mononuclear cells, human polymorphonuclear leukocytes, or both with a TIM receptor agonist.

69. A non-human animal generated by the method of any one of claims 3-68.

70. A non-human animal model, comprising two or more non-human animals of any one of claims 1, 2, 5-66, and 69, wherein the engraftment in each non-human animal is from a different human.

71. A method of generating a non-human animal model, comprising
for n individual non-human animals lacking endogenous mature T cells, engrafting a population of human mononuclear cells, human polymorphonuclear leukocytes, or both from a nth human into a nth non-human animal, wherein n is an integer ≥2,
thereby generating a non-human animal model comprising two or more individual non-human animals,
wherein the engraftments lack human cancer cells and produce a functional human immune system in the individual non-human animals.

72. A method of determining immunogenicity of an antigen or an immunogenic fragment thereof, or an antigenic therapy, comprising:
administering the antigen or the immunogenic fragment thereof, or the antigenic therapy to the one or more non-human animals of any one of claims 1, 2, 5-66, and 69; and
detecting an immune response to the antigen or the immunogenic fragment thereof, or the antigenic therapy in the one or more non-human animals.

73. The method of claim 72, further comprising predicting a clinical immunogenicity rate of the antigen or the immunogenic fragment thereof, or the antigenic therapy.

74. The method of claim 72 or 73, further comprising attributing the immunogenicity to a property of the antigen or an immunogenic fragment thereof, or the antigenic therapy.

75. A method of identifying an agent for modulating immune intolerance, comprising:
administering an antigen or an immunogenic fragment thereof, or an antigenic therapy to the one or more non-human animals of any one of claims 1, 2, 5-66, and 69;
administering an agent suspected to modulate immune intolerance of the one or more non-human animals; and
detecting an immune response to the antigen in the one or more non-human animals.

76. The method of any one of claims 72-75, wherein the antigen or an immunogenic fragment thereof, or antigenic therapy comprises a vaccine, a therapeutic, or both.

77. The method of claim 75 or 76, wherein the agent comprises a polynucleotide, a polypeptide, a cell, a tissue, or any combination of the foregoing.

78. The method of any one of claims 72-77, wherein detecting an immune response to the antigen or an immunogenic fragment thereof, or antigenic therapy comprises detecting antibodies to the antigen or an immunogenic fragment thereof, or antigenic therapy.

79. The method of any one of claims 72-78, wherein the one or more non-human animals comprise human CD20+ B cells.

80. The method of claim 79, wherein the human CD20+ B cells are present in the one or more non-human animals at a greater level after the administering an antigen or an immunogenic fragment thereof, or an antigenic therapy to the one or more non-human animals, as compared to before the administering.

81. The method of any one of claims 72-80, wherein the one or more non-human animals comprise two or more non-human animals.

82. The method of claim 81, wherein the engraftment in each non-human animal is from the same human.

83. The method of claim 81, wherein the engraftment in each non-human animal is from a different human.

84. A non-human animal, wherein the non-human animal:
a) lacks endogenous mature T cells, endogenous mature B cells, and endogenous mature NK cells;
b) comprises an engrafted population of human mononuclear cells, human polymorphonuclear leukocytes, or both, wherein the engraftment lacks human cancer cells, and wherein the population of human mononuclear cells comprises human T cells, human B cells, human NK cells, human monocytes, and human macrophages;
c) expresses human IL-3, human GM-CSF, and human SF;
d) lacks Rag1 expression, function, or both; and
e) lacks IL2rγ expression, function, or both.
